(19) United States
(12) Patent Application Publication
(10) Pub. No.: US 2025/0297286 A1
(43) Pub. Date: Sep. 25, 2025

(54) METHODS AND COMPOSITIONS FOR THE PRODUCTION OF RECOMBINANT ADENO-ASSOCIATED VIRUS (rAAV) VECTORS

(71) Applicant: BEACON THERAPEUTICS (USA), INC., Alachua, FL (US)

(72) Inventors: Dane Phelan, Alachua, FL (US); David R. Knop, Alachua, FL (US)

(21) Appl. No.: 19/061,630

(22) Filed: Feb. 24, 2025

Related U.S. Application Data
(63) Continuation of application No. PCT/US2023/031101, filed on Aug. 24, 2023.
(60) Provisional application No. 63/400,619, filed on Aug. 24, 2022.

(51) Int. Cl. C12N 15/864; C12N 5/10; C12N 7/04

(52) U.S. Cl. CPC C12N 15/8645; C12N 5/10; C12N 7/04; C12N 2510/02; C12N 2710/16631; C12N 2710/16643; C12N 2750/14143; C12N 2750/14152; C12N 2800/50

(57) ABSTRACT

The present disclosure provides methods and compositions for producing adeno-associated virus (rAAV) vectors.

### RELATED APPLICATIONS

The instant application is a continuation of International Application No. PCT/US2023/031101, filed on Aug. 24, 2023, which claims the benefit of priority to U.S. Provisional Application No. 63/400,619, filed on Aug. 24, 2022, the entire contents of each of which are incorporated herein by reference.

### SEQUENCE LISTING

The instant application contains a Sequence Listing, which has been submitted electronically in. XML format and is hereby incorporated by reference in its entirety. Said. XML copy, created on Jun. 12, 2025, is named “119561-20802.xml” and is 4,737 bytes in size. The sequence listing contained in this. XML file is part of the specification and is hereby incorporated by reference herein in its entirety.

### BACKGROUND

The use of recombinant adeno-associated viruses (rAAVs) vectors for in vivo gene therapy has recently been the focus of intense research, primarily because of rAA Vs safety profile which is characterized by non-pathogenicity to humans, low immunogenicity, and long-term gene expression, along with recent clinical breakthroughs. See e.g., Gimpel et al. (Analytical methods for process and product characterization of recombinant adeno-associated virus-based gene therapies. Mol Ther Methods Clin Dev. 17 (20): 740-754, 2021) and Escandell et al. (Leveraging rAAV bioprocess understanding and next generation bioanalytics development. Current Opinion in Biotechnology. 74:271-277, 2022). The U.S. Food and Drug Administration (FDA) recently approved two rAAV-based gene therapies and there are over 200 active clinical trials for rAAV-based gene therapies for treating a range of human diseases. As the clinical potential of rAAV is being recognized, the expected increase in demand for rAAV vectors poses challenges, for example, in the scale-up of production processes without compromising rAAV vector yield, potency, purity, and safety.

Accordingly, there remains a significant unmet clinical need for improved methods and compositions for the production of recombinant adeno-associated virus (rAAV) vectors.

### FIELD OF THE INVENTION

The present disclosure relates to methods and compositions for the production of recombinant adeno-associated virus (rAAV) vectors for use in gene therapy applications.

### SUMMARY

Disclosed herein are methods and compositions for producing recombinant adeno-associated virus (rAAV) vector-based gene therapies. The present disclosure is based, at least in part, on the surprisingly discovery that even small quantities of input rAAV can be expanded via coinfection alongside a chosen rep/cap rHSV vector, without requiring the generation, propagation, and deployment of a gene of interest (GOI) rHSV. The hybrid herpes-assisted vector expansion (“hybrid HAVE”) process described herein advantageously provides a robust, scalable, and highly productive method for manufacturing of rAAV vector for GOI cassettes which have to-date proven to be incompatible with vectorization into rHSV. The methods and compositions disclosed herein are particularly advantageous for manufacturing of rAAV vector for GOI cassettes encoding therapeutic agents (e.g., transmembrane proteins, e.g., light-sensing proteins) useful in optogenetic applications, such as optogenetic gene therapy, that are incompatible with vectorization with rHSV vectors. In particular embodiments, it has been surprisingly found that the methods and compositions disclosed herein improve the production of rAAV vector for GOI cassettes which have to-date proven to be incompatible with vectorization into rHSV. Such improvements provided by the hybrid HAVE process include, for example, increasing the total rAAV generated by harvest time and/or increasing the proportion of vector encapsidated with full length genomes, e.g., as compared to a reference process. In certain embodiments, the reference process may comprise a transfection-mediated production of the rAAV vector, such as by triple transfection.

Accordingly, the present disclosure provides methods and compositions for producing an rAAV vector including a transgene cassette comprising a nucleotide sequence encoding a gene of interest (GOI) that is incompatible with vectorization with rHSV vectors. In particular embodiments, the present disclosure provides methods and compositions for producing an rAAV vector including a transgene cassette comprising a nucleotide sequence encoding a gene of interest (GOI), e.g., that cannot be stably vectorized within rHSV. Accordingly, the present invention provides methods and compositions for producing recombinant adeno-associated viruses (rAAVs) vector-based gene therapies encoding various GOI, including those that cannot be stably vectorized within rHSV.

In one aspect, the present disclosure provides methods and compositions for producing rAAV vector for GOI cassettes encoding therapeutic agents (e.g., transmembrane proteins, e.g., light-sensing proteins) useful in optogenetic applications, such as optogenetic gene therapy, that are incompatible with vectorization with rHSV vectors.

Further, despite validation of recombinant adeno-associated viruses (rAAVs) vector-based gene therapy across multiple disease indications, rAAV manufacturing continues to pose a challenge to the development of therapeutically effective rAAV vectors capable of delivering genetic material safely into a subject's own cells. Indeed, the complexity of rAAV vector-based gene therapy manufacturing has historically resulted in poor reproducibility and lack of reliability, for example, in meeting material needs beyond the early human clinical setting. At least in part, rAAV vector manufacturing has been limited inter alia by inefficient constructs, poor scalability, inadequate yields, and insufficient purity to meet both clinical and commercial needs. The present disclosure, in its various embodiments, provides methods and compositions that overcome many of the known challenges associated with rAAV vector production, e.g., reduced rAAV vector yield, potency, purity, and safety.

Accordingly, in one aspect, the present disclosure provides methods and compositions for the production of rAAV vectors having many benefits previously unrealized with art known rAAV vector production processes. Without wishing to be bound by theory, use of the methods and compositions described herein may achieve increased production efficiency (e.g., increased rAAV vector yield, potency, purity, and safety) with reduced manufacturing costs, e.g., as compared to art known rAAV vector production processes. In addition, still further benefits for the production of rAAV vectors, and their use in gene therapy applications, flow from the methods and compositions described herein, as will be apparent to one of ordinary skill in the art upon reading the present disclosure.

Accordingly, in one aspect, the present disclosure provides a method for producing recombinant adeno-associated virus (rAAV) vectors comprising the steps of: (i) providing a cell; (ii) infecting the cell with a recombinant herpes simplex virus (rHSV) vector comprising a nucleotide sequence encoding a rep/cap gene cassette, wherein the rep/cap gene cassette comprises the adeno-associated virus serotype (AAV) rep gene and a recombinant cap gene encoding structural proteins for a predetermined capsid serotype; (iii) infecting the cell with a recombinant adeno-associated virus (rAAV) seed vector comprising a nucleotide sequence encoding a transgene cassette, wherein the transgene cassette comprises a gene of interest (GOI); (iv) culturing the cell under conditions that allow production of rAAV; and (v) collecting the rAAV produced.

Accordingly, in one aspect, the present disclosure provides a method for producing recombinant adeno-associated virus (rAAV) vectors comprising the steps of: (i) providing a cell; (ii) infecting the cell with a recombinant herpes simplex virus (rHSV) vector comprising a nucleotide sequence encoding a rep/cap gene cassette, wherein the rep/cap gene cassette comprises the adeno-associated virus serotype 2 (AAV2) rep gene and a recombinant cap gene encoding structural proteins for a predetermined capsid serotype; (iii) infecting the cell with a recombinant adeno-associated virus (rAAV) seed vector comprising a nucleotide sequence encoding a transgene cassette, wherein the transgene cassette comprises a gene of interest (GOI); (iv) culturing the cell under conditions that allow production of rAAV; and (v) collecting the rAAV produced. In general, the AAV seed vector comprises an rAAV manufacturing reagent as opposed, e.g., to an rAAV product of the manufacturing.

In one aspect, the present disclosure provides methods of expressing a rep gene (e.g., an adeno-associated virus serotype 2 (AAV2) rep gene) and/or a cap gene (e.g., a recombinant cap gene encoding structural proteins for a predetermined capsid serotype). In some embodiments, the rep gene is encoded by a nucleotide sequence operably linked to a nucleotide sequence encoding a promoter, e.g., a native (wild-type) promoter. In some embodiments, the recombinant cap gene is encoded by a nucleotide sequence operably linked to a nucleotide sequence encoding a promoter, e.g., a native (wild-type) promoter.

In some embodiments, the predetermined capsid serotype comprises an AAV serotype selected from the group consisting of AAV-1, AAV-2, AAV-2tYF, AAV-3, AAV-3b, AAV-4, AAV-5, AAV-6, AAV-7, AAV-8, and AAV-9, or variants thereof. In some embodiments, the AAV serotype is AAV-1. In some embodiments, the AAV serotype is AAV-2. In some embodiments, the AAV serotype is AAV-2tYF. In some embodiments, the AAV serotype is AAV-3. In some embodiments, the AAV serotype is AAV-3b. In some embodiments, the AAV serotype is AAV-4. In some embodiments, the AAV serotype is AAV-5. In some embodiments, the AAV serotype is AAV-6. In some embodiments, the AAV serotype is AAV-7. In some embodiments, the AAV serotype is AAV-8. In some embodiments, the AAV serotype is AAV-9. For example, the predetermined capsid serotype may be AAV-2 or AAV-2tYF.

In some embodiments, (i) the rep/cap gene cassette comprises an AAV rep gene serotype selected from the group consisting of AAV-1, AAV-2, AAV-2tYF, AAV-3, AAV-3b, AAV-4, AAV-5, AAV-6, AAV-7, AAV-8, and AAV-9, or variants thereof; (ii) the rep/cap gene cassette comprises a adeno-associated virus serotype 2 (AAV2) rep gene; (iii) the predetermined capsid serotype comprises an AAV serotype selected from the group consisting of AAV-1, AAV-2, AAV-2tYF, AAV-3, AAV-3b, AAV-4, AAV-5, AAV-6, AAV-7, AAV-8, and AAV-9, or variants thereof; (iv) the predetermined capsid serotype is tropic for ocular cells, retinal cells, retinal pigment epithelium, and/or photoreceptors, optionally AAV2.7m8; (v) the predetermined capsid serotype is AAV-2 or AAV-2tYF; and/or (vi) the predetermined capsid serotype is a non-naturally occurring, synthetic, or engineered capsid.

In some embodiments, the rHSV vector is replication defective. In some embodiments, the nucleotide sequence encoding the rep/cap gene cassette is integrated into the locus of the thymidine kinase gene of the rHSV vector. In some embodiments, the rHSV vector is a HSV-1 vector. In some embodiments, the AAV rep gene is encoded by a nucleotide sequence operably linked to a nucleotide sequence encoding a promoter. In some embodiments, the AAV2 rep gene is encoded by a nucleotide sequence operably linked to a nucleotide sequence encoding a promoter. In some embodiments, the recombinant cap gene is encoded by a nucleotide sequence operably linked to a nucleotide sequence encoding a promoter. In some embodiments, each promoter is independently a native (wild-type) promoter. In some embodiments, each promoter is independently an endogenous promoter or is a heterologous promoter, optionally wherein each promoter is independently a native AAV promoter.

In some embodiments, the rAAV seed vector comprises a transgene cassette. In some embodiments, the transgene cassette comprises a nucleotide sequence encoding a gene of interest (GOI).

In some embodiments, the GOI is encoded by a nucleotide sequence operably linked to a nucleotide sequence encoding a promoter. In some embodiments, the promoter is a native (wild-type) promoter. In some embodiments, the promoter is an endogenous GOI promoter or is a heterologous promoter, optionally a chicken beta-actin (CBA) promoter.

In some embodiments, the rAAV seed vector comprises a transgene cassette or an additional transgene cassette comprising a nucleotide sequence encoding a reporter molecule, such as a green fluorescent protein. In some embodiments, the reporter molecule is encoded by a nucleotide sequence operably linked to a nucleotide sequence encoding a promoter, optionally wherein the reporter molecule is selected from the group consisting of beta-galactosidase, neomycin phosphoro-transferase, chloramphenicol acetyl transferase, thymidine kinase, luciferase, beta-glucuronidase, xanthine-guanine phosphoribosyl transferase, and green fluorescent protein.

In some embodiments, the transgene cassette and/or the additional transgene cassette is flanked by AAV inverted terminal repeats (ITRs). In some embodiments, the transgene cassette and/or the additional transgene cassette is flanked by AAV2 inverted terminal repeats (ITRs). In some embodiments, the transgene cassette and/or the additional transgene cassette is terminated by a SV40 polyadenylation signal or a bovine growth hormone polyadenylation signal. In some embodiments, the GOI encodes a protein. In some embodiments, the GOI encodes a therapeutic RNA, such as an antisense RNA or a siRNA or an miRNA, or encodes a gene editing reagent, such as a guide nucleic acid and/or a nuclease. In some embodiments, the GOI encodes a therapeutic protein. In some embodiments, the GOI is codon-optimized for human expression. In some embodiments, the GOI is incompatible with vectorization with rHSV vectors (e.g., cannot be inserted into and/or expressed from the viral genome), optionally wherein: (i) the GOI cannot be stably vectorized within rHSV; and/or (ii) the GOI cannot be sufficiently expressed from the HSV genome In some embodiments, and in particular in some embodiments in which the GOI encodes a protein that is incompatible with vectorization with rHSV vectors, the GOI encodes a transmembrane protein. In some embodiments, such a transmembrane protein may be a monotopic, a biotopic, or a polytopic integral transmembrane protein. The transmembrane protein may include one or more hydrophobic regions, which regions contain α-helical portions. The transmembrane protein may be, for example, a channel protein, such as an ion channel, or a carrier protein, and/or may have one or more of the following functions: receptor, receptor ligand, structural (e.g., beta-dystroglycan), adhesion, transport (e.g., ABC transporter, P-glycoprotein), and/or gene regulation. In some embodiments, the transmembrane protein is a G protein-coupled receptor (GPCR) such as a cadherin (calcium-dependent adhesion molecule) or an opsin, or is a G protein-coupled inwardly rectifying potassium channel (GIRK) such as GIRK-1,-2,-3 or-4. In some embodiments the transmembrane protein is a gap junction protein (GJP), such as a connexin (e.g., gap junction beta 2), and/or may have protein kinase activity, e.g., human insulin receptor.

In some embodiments the transmembrane protein may be a light-sensing protein useful in optogenetic applications, such as optogenetic gene therapy. Such a light-sensing protein may be an opsin, such as a microbial or vertebrate opsin (McClements et al (2020), Frontiers in Neuroscience, vol 14, article 570909). Such a microbial opsin may be channelrhodopsin-2 (ChR2) or an engineered variant thereof such as ReaChR or ChrimsonR, halorhodopsin (NpHR) or enhanced halorhodopsin (eNpHR) or Jaws. Such a vertebrate opsin may be rhodopsin (RHO), short-wave cone opsin (SWC), medium-wave cone opsin (MWC), long-wave cone opsin (LWC), melanopsin (OPN4), or an engineered opsin such as Chronos (ChR90) or multicharacteristic (polychromatic) opsin (MCO; e.g. U.S. Pat. No. 11,180,537).

In certain embodiments, the GOI encodes: (i) a membrane protein, optionally wherein the membrane protein is selected from the group consisting of an integral membrane protein, a transmembrane protein, a peripheral membrane protein, a lipid-anchored protein, a portion thereof, and combinations thereof; (ii) a transmembrane protein, optionally wherein the transmembrane protein comprises a light-sensing protein useful in optogenetic applications and/or optogenetic gene therapy; (iii) a transmembrane protein, optionally wherein the transmembrane protein is selected from the group consisting of a channel protein, an ion channel protein, a transport protein, a receptor protein, a kinase protein, an adhesion protein, a structural protein, a G protein-coupled receptor, a G protein-coupled inwardly rectifying potassium channel (GIRK), a gap junction protein, a cadherin, a connexin, an opsin, a portion thereof, and combinations thereof; (iv) an opsin, optionally wherein the opsin is selected from the group consisting of a channelrhodopsin-2 (ChR2) or an engineered variant thereof, optionally a ReaChR or ChrimsonR, a halorhodopsin (NpHR), an enhanced halorhodopsin (eNpHR), a Jaws, a rhodopsin (RHO), a short-wave cone opsin (SWC), a medium-wave cone opsin (MWC), a long-wave cone opsin (LWC), melanopsin (OPN4), an engineered opsin, optionally a Chronos (ChR90) or a multicharacteristic (polychromatic) opsin (MCO); (v) a fusion protein; and/or (vi) a therapeutic agent, optionally a therapeutic protein.

In some embodiments, the infection of the cell with the rHSV vector is performed before infection of the cell with the seed rAAV vector. In some embodiments, the infection of the cell with the rHSV vector is performed after infection of the cell with the seed rAAV vector. In some embodiments, the infection of the cell with the rHSV vector is performed at about the same time as infection of the cell with the seed rAAV vector.

In some embodiments, the multiplicity of infection (MOI) (i.e., the ratio of the virus to the cells) of the rHSV vector is from about 1 to about 4 (e.g., about 1, about 2, about 3, or about 4).

In some embodiments, the multiplicity of infection (MOI) of the seed rAAV vector is from about 1 to about 1,000 (e.g., about 1, about 2, about 3, about 4, about 5, about 6, about 7, about 8, about 9, about 10, about 20, about 30, about 40, about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, or about 1,000).

In some embodiments, the MOI configurations for the rHSV vector and the rAAV vector is about 4±2 and about 100±2 logs for the respective vectors. In some embodiments, the MOI configurations for the rHSV vector and the rAAV vector may be about 2 and about 30, about 2 and about 55, about 2 and about 100, about 2 and about 300, about 3 and about 30, about 3 and about 55, about 4 and about 1, about 4 and about 10, about 4 and about 30, about 4 and about 55, about 4 and about 100, about 4 and about 180, about 4 and about 315, about 4 and about 1000, about 5 and about 30, about 5 and about 55, about 5 and about 100, about 5 and about 180, or about 5 and about 315. In certain embodiments, the MOI configurations for the rHSV vector and the rAAV vector is about 2 and about 100.

In some embodiments, the method results in less than about 10,000 infectious particles being produced by the infected cell. In some embodiments, the method results in at least about 500 to about 10,000 infectious particles being produced by the infected cell. In some embodiments, the method results in at least about 500 to about 20,000 infectious particles being produced by the infected cell. In some embodiments, the method results in at least about 20,000 or more infectious particles being produced by the infected cell. In some embodiments, the method results in at least about 500, at least about 5000, at least about 10000, or at least about 20000, at least about 25000, at least about 30000, at least about 35000, at least about 40000, at least about 45000, at least about 50000, at least about 55000, at least about 60000, at least about 65000, at least about 70000, at least about 75000, at least about 80000, at least about 85000, at least about 90000, at least about 95000, at least about 100000 or more infectious rAAV particles being produced by the infected cell. In some embodiments, the infectious particles may be measured by, for example, an IC50 assay and/or qPCR. In some embodiments, the productivity in encapsidated genomes may be measured by, for example, qPCR. In some embodiments, the infectious particles comprise the total output particles per input cells. In some embodiments, the infectious particles comprise the mean number of particles per each cell in the infected culture.

In some embodiments, the seed rAAV vector can be produced by any rAAV vector production process. In some embodiments, the seed rAAV vector can be produced by a hybrid herpes-assisted vector expansion (HAVE) process and/or a transfection-based process.

In some embodiments, the method may further comprise refeeding for recursive expansion.

Accordingly, in one aspect, the present disclosure provides a kit for producing rAAV comprising: (i) a recombinant herpes simplex virus (rHSV) vector comprising a nucleotide sequence encoding a rep/cap gene cassette, wherein the rep/cap gene cassette comprises the adeno-associated virus serotype (AAV) rep gene and a recombinant cap gene encoding structural proteins for a predetermined capsid serotype; (ii) a recombinant adeno-associated virus (rAAV) seed vector comprising a nucleotide sequence encoding a transgene cassette, wherein the transgene cassette comprises a gene of interest (GOI); and (iii) instructions for use.

Accordingly, in one aspect, the present disclosure provides a kit for producing rAAV comprising: (i) a recombinant herpes simplex virus (rHSV) vector comprising a nucleotide sequence encoding a rep/cap gene cassette, wherein the rep/cap gene cassette comprises the adeno-associated virus serotype 2 (AAV2) rep gene and a recombinant cap gene encoding structural proteins for a predetermined capsid serotype; (ii) a recombinant adeno-associated virus (rAAV) seed vector comprising a nucleotide sequence encoding a transgene cassette, wherein the transgene cassette comprises a gene of interest (GOI); and (iii) instructions for use.

Accordingly, in one aspect, the present disclosure provides a cell, comprising: (i) a recombinant herpes simplex virus (rHSV) vector comprising a nucleotide sequence encoding a rep/cap gene cassette, wherein the rep/cap gene cassette comprises the adeno-associated virus serotype (AAV) rep gene and a recombinant cap gene encoding structural proteins for a predetermined capsid serotype; and (ii) a recombinant adeno-associated virus (rAAV) seed vector comprising a nucleotide sequence encoding a transgene cassette, wherein the transgene cassette comprises a gene of interest (GOI). In some embodiments, the cell is a mammalian cell, such as a BHK cell, a Vero cell, a HEK293 cell, or a HeLa cell.

Accordingly, in one aspect, the present disclosure provides a cell, comprising: (i) a recombinant herpes simplex virus (rHSV) vector comprising a nucleotide sequence encoding a rep/cap gene cassette, wherein the rep/cap gene cassette comprises the adeno-associated virus serotype 2 (AAV2) rep gene and a recombinant cap gene encoding structural proteins for a predetermined capsid serotype; and (ii) a recombinant adeno-associated virus (rAAV) seed vector comprising a nucleotide sequence encoding a transgene cassette, wherein the transgene cassette comprises a gene of interest (GOI). In some embodiments, the cell is a mammalian cell, such as a BHK cell, a Vero cell, a HEK293 cell, or a HeLa cell.

Using the recombinant herpesvirus (virion) or vector according to the invention, which may comprise the helper functions which are required for AAV replication and may provide an adequate quantity of Rep and Cap proteins, it is possible to replicate AAV vectors by infecting eukaryotic cells, including a variety of generally available cell lines. The invention furthermore relates, therefore, to a cell which contains a rHSV vector and a rAAV vector as described herein. The cell is preferably a mammalian cell, in particular a cell which can be cultured on a permanent basis. Examples are rodent cells, such as BHK cells or derivatives thereof, e.g., BHK21 or sBHK. However, it is also possible to use any other cells capable of being infected by HSV1 and AAV, e.g., primate cells such as Vero cells, HEK293, or HeLa cells. The cell can contain the virus, the vector or the virion in extrachromosomal form, in one or more copies. Cells of this nature can, for example, be obtained by infection.

The invention also relates to an improved process for producing infectious rAAV vector preparations, which can reproducibly achieve yields of vector in quantities within an order of magnitude of that from analogous established processes.

### BRIEF DESCRIPTION OF THE DRAWINGS

FIGS. 1A-1D show exemplary process flow diagrams (PFDs) illustrating the hybrid herpes-assisted vector expansion (HAVE) process and nearest alternative recombinant adeno-associated virus (rAAV)-manufacturing schemata. Individual bioprocessing executions are shown in gray boxes. Dashed gray boxes illustrate, for clarity, prospective means of generating starting biologics and are not considered a component of the overarching process, but rather are included as non-exclusive examples.

FIG. 1A shows generalized PFD of the existing herpes-assisted vector expansion (HAVE) platform, currently used by AGTC for manufacture of clinical trial drug product. A rep/cap-encoding rHSV vector (dark-shaded round virion) and a gene of interest (GOI)-encoding rHSV vector (light-shaded round virion) are propagated in a complementing culture (e.g., the adherent V27 cell line, light-shaded right-facing T-flask) to establish banks that are subsequently coinfected into a bioreactor culture (e.g. containing a suspension BHK cell line, light-shaded Erlenmeyer flask), to produce rAAV ultimately processed to drug product (large dark-shaded icosahedral virion). Initial generation of rHSV vector can be, for example, obtained by established cotransfection/recombineering procedures.

FIG. 1B shows a generalized process flow diagram (PFD) of the hybrid HAVE process. Initial GOI-encoding rAAV (“seed rAAV”, small dark-shaded icosahedral virion) can be obtained through various means, here illustrated by triple transfection at small scale, as an example. Existing rAAV of any capsid serotype is expanded via coinfection of cultures (e.g., bioreactors of sub-manufacturing scale) with an rHSV vector encoding a cap gene that determines the desired capsid serotype of the rAAV product bank. Seed rAAV vector can be expanded recursively to arbitrary passage number and scale before the terminal production processed to drug product.

FIG. 1C shows a simplified process flow diagram (PFD) of a traditional triple transfection process for comparison. Each of up to three bacterial cultures (dark-shaded Erlenmeyer flasks) containing rep/cap-encoding, helper gene-encoding, or GOI-encoding plasmids are expanded from bacterial banks and processed to generate plasmids for use in manufacture of drug product (rings with colored shading).

FIG. 1D shows a diagrammatic relationship communicating relative productivity and versatility advantages of the three processes: HAVE (FIG. 1A), hybrid HAVE (FIG. 1B), and traditional triple transfection (FIG. 1C).

FIGS. 2A-2B show a pilot demonstration of the hybrid HAVE process in a small-scale test model. Cultures of suspension BHK cells at a volume of 40 mL were infected with the rHSV-rep2cap2t YF vector at an MOI of 4 and either the rHSV-CBA-hGFP vector at an MOI of 1 (conventional HAVE) or the rAAV2tYF-CBA-hGFP vector at MOIs spanning a 4-log range (hybrid HAVE). The following day, cultures were lysed via detergent and centrifuged to remove large cell debris, and rAAV in the lysate was quantified via a qPCR assay for DNase-resistant particles (DRP).

FIG. 2A shows a bar graph of cellular rAAV productivity (DNase-resistant particles (DRP)/cell) with respect to multiplicity of infection (MOI) at 24-hours and 48-hours harvest time (hpi). Error bar lengths represent twice the standard deviation from the mean for duplicate biological replicates.

FIG. 2B shows a bar graph of AAV Expansion Factor (output DRP/input DRP) with respect to multiplicity of infection (MOI) at 24-hours and 48-hours harvest time (hpi). Results are shown in the ratio of input rAAV vector to output rAAV vector to emphasize that even at low productivity conditions, e.g. MOI configurations that would be suboptimal for manufacturing, small quantities of rAAV in sample material can be rapidly amplified.

FIGS. 3A-3B show a demonstration of the rHSV-incompatibility of some therapeutically relevant GOIs, limiting their use with conventional HAVE, and the present invention's ability to circumvent this obstacle.

FIG. 3A shows a bar graph of rHSV plaques relative to control with respect to different cotransfected overexpression constructs, including: humanized green fluorescent protein (hGFP), complement factor H (CFH), retinitis pigmentosa GTPase regulator (RPGR), cyclic nucleotide gated channel beta 3 (CNGB3), ATP binding cassette subfamily D member 1 (ABCD1), channel rhodopsin and GFP fusion protein (ChR-GFP), and a gap junction protein (GJP). In simulation of the upstream-most step in the creation of rHSV vectors, V27 cells in wells of a 24-well plate were simultaneously transfected using the TransIT-X2 transfection reagent with 200 ng of genomic DNA prepared from an mCherry-encoding rHSV vector and 300 ng of each of various plasmids in which a chosen GOI was placed between the CBA promoter and the rBG polyadenylation signal, and overlain the following day with medium containing 0.2% human IgG to prevent the formation of secondary plaques. Six days after transfection, plaques were stained and counted. Green fluorescent protein (GFP) is used as a control for reference, complement factor H (CFH) is representative of a transgene non-problematic with rHSV vectors, retinitis pigmentosa GTPase regulator (RPGR) and cyclic nucleotide gated channel beta 3 (CNGB3) are representative of transgenes which can be otherwise accommodated into rHSV vectors with modifications to the cassette design (e.g., use of cell-specific promoters not active during vector production), and ATP binding cassette subfamily D member 1 (ABCD1), a channel rhodopsin and GFP fusion protein (ChR-GFP), and a gap junction protein (GJP) are examples of vectors which have not been able to be incorporated into rHSV vectors. Error bar lengths represent twice the standard deviation from the mean for triplicate biological replicates.

FIG. 3B shows a bar graph of cellular rAAV productivity (DNase-resistant particles (DRP)/cell) with respect to the gene of interest (GOI), humanized green fluorescent protein (hGFP) and channel rhodopsin and GFP fusion protein (ChR-GFP). Specifically, FIG. 3B shows a demonstration of hybrid HAVE for the production of a control rAAV product (e.g., hGFP) and for the production of an otherwise problematic transmembrane GOI-encoding rAAV product at small-scale (e.g., ChR-GFP). Cultures of suspension BHK cells at a scale of 40 mL were infected with the rHSV-rep2cap2tYF vector at an MOI of 4 and the indicated GOI vector, at an MOI of 1 for the rHSV control or 100 for the rAAV. The following day, cultures were lysed via detergent and centrifuged to remove large cell debris the following day, after which vector product was quantified via a qPCR assay for DNase-resistant particles (DRP). Titered clarified lysate from an initial production using triple transfection-procured rAAV seed vector was subsequently used for a second round of expansion without significant compromise in product yield. Error bar lengths represent twice the standard deviation from the mean for duplicate biological replicates.

FIGS. 4A-4B show a demonstration of the robustness of the hybrid HAVE process at small-scale with respect to rAAV capsid serotype produced and process intermediate used as seed vector.

FIG. 4A shows a bar graph of cellular rAAV productivity (DNase-resistant particles (DRP)/cell) with respect to rAAV capsid serotype produced, including: rAAV1, rAAV2, rAAV2tYF, rAAV3b, rAAV5, rAAV8, and rAAV9. To demonstrate the ability of hybrid HAVE to produce a multitude of rAAV capsid serotypes, cultures of suspension BHK cells at a volume of 40 mL were infected with the GOI seed vector rAAV2tYF-CBA-hGFP at an MOI of 100 and each of various rep/cap rHSV stocks encoding the AAV2 rep gene and each of the indicated cap genes at an MOI of 4. The following day, cultures were lysed via detergent and centrifuged to remove large cell debris the following day, after which vector product was quantified via a qPCR assay for DNase-resistant particles (DRP). Error bar lengths represent twice the standard deviation from the mean for duplicate biological replicates.

FIG. 4B shows a graph of cellular rAAV productivity (DRP)/cell) with respect to multiplicity of infection (MOI) configuration (rep/cap rHSV: GOI rAAV). To demonstrate hybrid HAVE's insensitivity to purification intermediates, rAAV production was done as for the preceding panel, but using a GJP-encoding seed vector production configuration (MOI of 2 with rHSV-rep2capSV6d and MOI of 100, or a roughly half-log offset therefrom, with the GJP-encoding rAAV seed vector), with the seed vector comprised of P1 triple transfection-produced iodixanol gradient-purified rAAV, P2 hybrid HAVE-produced (using the aforementioned P1 rAAV) affinity column-purified rAAV (enriched for rAAV particles over other biomolecules relative to clarified lysate), or the same vector purified further through anion-exchange column (enriched for fully packaged rAAV particles over empty or capsids packaged with sub-genomic length DNA). Error bar lengths represent twice the standard deviation from the mean for duplicate biological replicates.

FIGS. 5A-5B shows an exemplary hybrid HAVE process for manufacturing of a GJP-encoding rAAV gene therapy product in bioreactor-format at variable scale.

FIG. 5A shows a bar graph of cellular rAAV productivity (DNase-resistant particles (DRP)/cell) with respect to bioreactor scale, e.g., at 1 L, 3 L, 10 L, and 50 L. Productions were executed in bioreactors of various formats, and cellular rAAV productivity was determined based on clarified lysate titers before processing to drug product. Error bar lengths represent twice the standard deviation from the mean for mean performances from each independent run (not including as replicates replicate bioreactors within each run).

FIG. 5B shows a pie chart with three components: empty capsids (“empty”), capsids packaged with sub-vector-length DNA (“partial”), and capsids packaged with vector length DNA (full), with respect to rHSV multiplicity of infection (MOI). The impact of the rep/cap-encoding rHSV vector MOI was assessed by evaluating parallel 3 L productions with MOI configurations for rHSV and rAAV of 4 and 100 against 2 and 100, in duplicates for each. In addition to titers, profiles of rAAV packaging efficiency were compared via analytical ultracentrifugation on intermediates processed through affinity column and anion-exchange column purification steps, where proportions of empty capsids, capsids packaged with sub-vector-length DNA (“partial”), and capsids packaged with vector length DNA (full) were estimated from sedimentation analysis. Reduction of the MOI, and concomitant reduction in the depletion rate of requisite rHSV banks used for rAAV manufacture, was achieved without compromise in vector quality.

FIG. 6 shows a bar graph of total rAAV yield, as determined from qPCR titration of DNase-resistant particles within the bulk harvest material, from 3 L bioreactor-format productions of the AGTC-501 vector using distinct rAAV production processes. Error bar lengths represent twice the standard deviation from the mean for duplicate biological replicates.

### DETAILED DESCRIPTION

### I. Definitions

In order that the present disclosure may be more readily understood, certain terms are first defined.

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately” usually means within 5%, or more preferably within 1%, of a given value or range.

As used herein, the term “bioreactor” is meant to refer broadly to any apparatus that can be used for the purpose of culturing cells.

As used herein, the terms “gene” or “coding sequence,” is meant to refer broadly to a nucleic acid sequence, such as a DNA region (e.g., the transcribed region), which encodes a protein or portion thereof. A coding sequence is transcribed (DNA) and translated (RNA) into a polypeptide when placed under the control of an appropriate regulatory region, such as a promoter. A gene may comprise several operably linked fragments, such as a promoter, a 5′-leader sequence, a coding sequence and a 3′-non-translated sequence, comprising a polyadenylation site. The phrase “expression of a gene” refers to the process wherein a gene is transcribed into an RNA and/or translated into a protein or portion thereof (e.g., a functional protein or portion thereof).

As used herein, the term “gene of interest (GOI),” refers broadly to a heterologous sequence introduced into an AAV expression vector, and typically refers to a nucleic acid sequence encoding, e.g., a protein of therapeutic use in a subject, e.g., a human or animal subject. In some embodiments, the GOI encodes a therapeutic agent, a peptide, a polypeptide, a protein, a fusion protein, an oligonucleotide, a DNA molecule, an RNA molecule, an RNAi molecule, a small interfering RNA (siRNA), a short hairpin RNA (shRNA), a microRNA (miRNA), an antisense RNA (asRNA), a gene editing reagent, a guide sequence for a gene editing enzyme (e.g., a guide RNA (gRNA)), a gene editing enzyme (e.g., a nuclease), and/or any combination thereof.

In some embodiments, the GOI is incompatible with vectorization with rHSV vectors (e.g., cannot be inserted into and/or expressed from the viral genome). In some embodiments, and in particular in some embodiments in which the GOI encodes a protein that is incompatible with vectorization with rHSV vectors, the GOI encodes a membrane protein, such as a transmembrane protein. In some embodiments, the transmembrane protein may be a light-sensing protein useful in optogenetic applications, such as optogenetic gene therapy.

In some embodiments, the GOI encodes a membrane protein or a portion thereof. In some embodiments, the GOI encodes a membrane protein selected from the group consisting of an integral membrane protein, a transmembrane protein, a peripheral membrane protein, a lipid-anchored protein, a portion thereof, and a combination thereof.

In some embodiments, the GOI encodes a therapeutic agent, such as a therapeutic protein. In some embodiments, the GOI encodes a fusion protein or a portion thereof.

As used herein, the term “fusion protein” refers to a protein composed of two or more polypeptides that, although typically not joined in their native state, are joined by their respective amino and carboxyl termini through a peptide linkage to form a single continuous polypeptide, it is understood that the two or more polypeptide components can either be directly joined or indirectly joined, e.g., through a peptide linker or spacer.

As used herein, the term “membrane protein” refers to any protein that attaches to, inserts into, is a part of, or otherwise associates with a lipid or biological membrane, such as a membrane of a cell or an organelle. Exemplary membrane proteins include, without limitation, integral membrane proteins, which can be a permanent part of the cell membrane and can either penetrate the membrane (referred to as “integral polytopic proteins” or “transmembrane proteins”) or can be associated with one or the other side of the membrane (referred to as “integral monotopic proteins”), and peripheral membrane proteins, which can be transiently associated with the cell membrane. The interaction between an integral monotopic protein and a cell membrane may be mediated, e.g., by (i) an amphipathic α-helix parallel to the membrane plane; (ii) a hydrophobic loop; (iii) a covalently bound membrane lipid; and/or (iv) an electrostatic and/or ionic interaction with a membrane lipid, such as through a calcium ion. Peripheral membrane proteins may be attach to integral membrane proteins and/or may penetrate the peripheral regions of the lipid bilayer. Membrane proteins also include lipid-anchored proteins, which are proteins located on the surface of the cell membrane that are covalently attached to lipids embedded within the cell membrane. Exemplary lipid-anchored proteins include, without limitation, prenylated proteins, fatty acylated proteins, and glycosylphosphatidylinositol-linked proteins.

As used herein, the terms “herpesvirus” or “herpesviridae family”, are meant to refer broadly to the general family of enveloped, double-stranded DNA viruses with relatively large genomes. The family replicates in the nucleus of a wide range of vertebrate and invertebrate hosts, in preferred embodiments, mammalian hosts, for example in humans, horses, cattle, mice, and pigs. Exemplary members of the herpesviridae family include cytomegalovirus (CMV), herpes simplex virus types 1 and 2 (HSV1 and HSV2) and varicella zoster (VZV) and Epstein Barr Virus (EBV).

As used herein, the term “infection,” is meant to refer broadly to delivery of heterologous nucleic acids, such as DNA, into a cell by a virus. The term “co-infection” as used herein means “simultaneous infection,” “double infection,” “multiple infection,” or “serial infection” with two or more viruses. Infection of a producer cell with two (or more) viruses will be referred to as “co-infection.” The term “transfection” refers to a process of delivering heterologous nucleic acids to a cell by physical or chemical methods, such as plasmid DNA, which is transferred into the cell by means of electroporation, calcium phosphate precipitation, or other methods well known in the art.

As used herein, the term “recombinant” can refer to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, the term “recombinant HSV,” “rHSV,” and “rHSV vector,” is meant to refer broadly to isolated, genetically modified forms of herpes simplex virus type 1 (HSV) containing heterologous genes incorporated into the viral genome. As used herein, the term “rHSV-rep2cap2” or “rHSV-rep2cap1” refers to an rHSV in which the AAV rep and cap genes from either AAV serotype 1 or 2 have been incorporated into the rHSV genome. In certain embodiments, a DNA sequence encoding a therapeutic gene of interest has been incorporated into the viral genome.

As used herein, the term “AAV” is an abbreviation for adeno-associated virus, and may be used to refer to the virus itself or derivatives thereof, e.g., AAV vectors, AAV virus particles, and/or AAV virions. The term covers all subtypes and both naturally occurring and recombinant forms, except where required otherwise.

As used herein, the term “AAV virion” or “AAV virus” or “AAV viral particle” or “AAV vector particle” is meant to refer broadly to a complete virus particle, such as for example a wild type AAV virion particle, which comprises single stranded genome DNA packaged into AAV capsid proteins. The single stranded nucleic acid molecule is either sense strand or antisense strand, as both strands are equally infectious. The term “rAAV viral particle” refers to a recombinant AAV virus particle, e.g., a particle that is infectious but replication defective. A rAAV viral particle comprises single stranded genome DNA packaged into AAV capsid proteins.

As used herein, the term “therapeutic agent” refers to any agent that, when administered to a subject, has a beneficial effect. The term “therapeutic agent” refers to any agent that, when administered to a subject, has a therapeutic, diagnostic, and/or prophylactic effect and/or elicits a desired biological and/or pharmacological effect. In some embodiments, the therapeutic agent comprises a peptide, a polypeptide, a protein, a fusion protein, an oligonucleotide, a DNA molecule, an RNA molecule, an RNAi molecule, a small interfering RNA (siRNA), a short hairpin RNA (shRNA), a microRNA (miRNA), an antisense RNA (asRNA), a gene editing reagent, a guide sequence for a gene editing enzyme (e.g., a guide RNA (gRNA)), a gene editing enzyme (e.g., a nuclease), and/or any combination thereof. In some embodiments, the therapeutic agent comprises a therapeutic protein. In some embodiments, the therapeutic agent comprises a membrane protein. In some embodiments, the therapeutic agent comprises a membrane protein selected from the group consisting of an integral membrane protein, a transmembrane protein, a peripheral membrane protein, a lipid-anchored protein, a portion thereof, and combinations thereof. In some embodiments, the transmembrane protein may be a light-sensing protein useful in optogenetic applications, such as optogenetic gene therapy. In some embodiments, the therapeutic agent comprises a fusion protein or a portion thereof.

The term “therapeutic protein” as used herein refers to a protein, which has a therapeutic effect on a disease or disorder to be treated. The therapeutic protein, when expressed in an effective amount (or dosage) is sufficient to prevent, correct and/or normalize an abnormal physiological response. For example, a therapeutic protein may be sufficient to reduce by at least about 30 percent, more preferably by at least 50 percent, most preferably by at least 90 percent, a clinically significant feature of disease or disorder. In some embodiments, the therapeutic protein comprises a membrane protein or a portion thereof. In some embodiments, the therapeutic protein comprises a membrane protein selected from the group consisting of an integral membrane protein, a transmembrane protein, a peripheral membrane protein, a lipid-anchored protein, a portion thereof, and a combination thereof. In some embodiments, the transmembrane protein may be a light-sensing protein useful in optogenetic applications, such as optogenetic gene therapy. In some embodiments, the therapeutic protein comprises a fusion protein or a portion thereof.

As used herein, the term “transgene” is meant to refer to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

As used herein, a “transgene expression cassette” or “expression cassette” comprises the gene sequences that a nucleic acid vector is to deliver to target cells. These sequences include the gene of interest (e.g., GOI nucleic acids or variants thereof), one or more promoters, and/or minimal regulatory elements. Use of the term “transgene” encompasses both introduction of the gene or gene cassette for purposes of correcting a gene defect in the cell, or altering the functions of the transduced and/or surrounding cells, and introduction of the gene or gene cassette into a producer cell for purposes of enabling the cell to produce rAAV. In certain embodiments, introducing the gene or gene cassette for the purposes of correcting a gene defect in the cell or altering the functions of the transduced and/or surrounding cells can be carried out by gene therapy.

As used herein, the term “heterologous,” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

As used herein, the term “inverted terminal repeat” or “ITR” sequence is meant to refer to relatively short sequences found at the termini of viral genomes which are in opposite orientation. An “AAV inverted terminal repeat (ITR)” sequence, a term well-understood in the art, is an approximately 145-nucleotide sequence that is present at both termini of the native single-stranded AAV genome. The outermost nucleotides of the ITR can be present in either of two alternative orientations, leading to heterogeneity between different AAV genomes and between the two ends of a single AAV genome.

As used herein, the term “isolated” molecule (e.g., an isolated nucleic acid or protein or cell) means it has been identified and separated and/or recovered from a component of its natural environment.

As used herein, the term “minimal regulatory elements” is meant to refer to regulatory elements that are necessary for effective expression of a gene in a target cell and thus should be included in a transgene expression cassette. Such sequences could include, for example, promoter or enhancer sequences, a polylinker sequence facilitating the insertion of a DNA fragment within a plasmid vector, and sequences responsible for intron splicing and polyadenlyation of mRNA transcripts.

As used herein, the term “non-naturally occurring” is meant to refer broadly to a protein, nucleic acid, ribonucleic acid, or virus that does not occur in nature. For example, it may be a genetically modified variant, e.g., cDNA or codon-optimized nucleic acid.

As used herein, a “nucleic acid” or a “nucleic acid molecule” is meant to refer to a molecule composed of chains of monomeric nucleotides, such as, for example, DNA molecules (e.g., cDNA or genomic DNA). A nucleic acid may encode, for example, a promoter, the gene of interest or portion thereof, and/or regulatory elements. A nucleic acid molecule can be single-stranded or double-stranded. A “GOI nucleic acid” refers to a nucleic acid that comprises the GOI gene or a portion thereof, or a functional variant of the GOI gene or a portion thereof. A functional variant of a gene includes a variant of the gene with minor variations such as, for example, silent mutations, single nucleotide polymorphisms, missense mutations, and other mutations or deletions that do not significantly alter gene function.

The asymmetric ends of DNA and RNA strands are called the 5′ (five prime) and 3′ (three prime) ends, with the 5′ end having a terminal phosphate group and the 3′ end a terminal hydroxyl group. The five prime (5′) end has the fifth carbon in the sugar-ring of the deoxyribose or ribose at its terminus. Nucleic acids are synthesized in vivo in the 5′- to 3′-direction, because the polymerase used to assemble new strands attaches each new nucleotide to the 3′-hydroxyl (—OH) group via a phosphodiester bond.

As used herein, the terms “operatively linked” or “operably linked” or “coupled” can refer to a juxtaposition of genetic elements, wherein the elements are in a relationship permitting them to operate in an expected manner. For instance, a promoter can be operatively linked to a coding region if the promoter helps initiate transcription of the coding sequence. There may be intervening residues between the promoter and coding region so long as this functional relationship is maintained.

As used herein, a “percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. An example of an alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

Similarly, “sequence homology”, as used herein, also refers to a method of determining the relatedness of two sequences. To determine sequence homology, two or more sequences are optimally aligned as described above, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 95% of the amino acid residues or nucleotides in the reference sequence must match or comprise a conservative substitution with another amino acid or nucleotide, or a number of amino acids or nucleotides up to 5% of the total amino acid residues or nucleotides, not including conservative substitutions, in the reference sequence may be inserted into the reference sequence.

As used herein, the term “pharmaceutical composition” or “composition” is meant to refer to a composition or agent described herein (e.g. a recombinant adeno-associated (rAAV) expression vector and/or an rAAV virion), optionally mixed with at least one pharmaceutically acceptable chemical component, such as, though not limited to carriers, stabilizers, diluents, dispersing agents, suspending agents, thickening agents, excipients and the like.

As used herein, the terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, a “promoter” is meant to refer to a region of DNA that facilitates the transcription of a particular gene. As part of the process of transcription, the enzyme that synthesizes RNA, known as RNA polymerase, attaches to the DNA near a gene. Promoters contain specific DNA sequences and response elements that provide an initial binding site for RNA polymerase and for transcription factors that recruit RNA polymerase. According to some embodiments, the promoter is highly specific for tissue-specific expression. According to some embodiments, the promoter is an endogenous (wild-type) GOI promoter. According to some embodiments, the promoter is a synthetic promoter.

As used herein, the terms “treatment” or “treating” a disease or disorder are meant to refer to alleviation of one or more signs or symptoms of the disease or disorder, diminishment of extent of disease or disorder, stabilized (e.g., not worsening) state of disease or disorder, preventing spread of disease or disorder, delay or slowing of disease or disorder progression, amelioration or palliation of the disease or disorder state, and remission (whether partial or total), whether detectable or undetectable. For example, a gene of interest, when expressed in an effective amount (or dosage) is sufficient to prevent, correct, and/or normalize an abnormal physiological response, e.g., a therapeutic effect that is sufficient to reduce by at least about 30 percent, more preferably by at least 50 percent, most preferably by at least 90 percent, a clinically significant feature of disease or disorder. “Treatment” can also refer to prolonging survival as compared to expected survival if not receiving treatment.

As used herein, the term “vector” of “vectorized” is meant to refer to a recombinant plasmid or virus that comprises a nucleic acid to be delivered into a host cell, either in vitro or in vivo. In certain embodiments, the term “vector” is meant a recombinant plasmid or viral construct used as a vehicle for introduction of transgenes into cells.

As used herein, the term “recombinant viral vector” is meant to refer to a recombinant polynucleotide vector comprising one or more heterologous sequences (i.e., nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

As used herein, the term “recombinant AAV vector (rAAV vector)” is meant to refer to a polynucleotide vector comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle)”. In certain embodiments, the AAV virus capsid is a variant AAV capsid as described herein.

As used herein, the term a “rAAV virus” or “rAAV viral particle” or “rAAV virion” is meant to refer to a viral particle composed of at least one AAV capsid protein and an encapsidated rAAV vector genome.

The term “variant” or “variants”, with regard to polypeptides, refers to a polypeptide sequence differing by at least one amino acid from a parent polypeptide sequence, also referred to as a non-variant polypeptide sequence. In some embodiments, the variant polypeptide differs by at least one amino acid substitution. Amino acids also include naturally occurring and non-naturally occurring amino acids as well as derivatives thereof. Amino acids also include both D and L forms.

The terms “tropism” and “transduction” are interrelated, but there are differences. The term “tropism” as used herein refers to the ability of an AAV vector or virion to infect one or more specified cell types, but can also encompass how the vector functions to transduce the cell in the one or more specified cell types; i.e., tropism refers to preferential entry of the AAV vector or virion into certain cell or tissue type(s) and/or preferential interaction with the cell surface that facilitates entry into certain cell or tissue types, optionally and preferably followed by expression (e.g., transcription and, optionally, translation) of sequences carried by the AAV vector or virion in the cell, e.g., for a recombinant virus, expression of the heterologous nucleotide sequence(s). In some embodiments, the AAV vector has tropism for ocular cells, retinal cells, retinal pigment epithelium, and/or photoreceptors. In some embodiments, the capsid proteins (e.g., encoded by a rep/cap gene cassette) have tropism for ocular cells, retinal cells, retinal pigment epithelium, and/or photoreceptors.

As used herein, the term “transduction” refers to the ability of an AAV vector or virion to infect one or more particular cell types; i.e., transduction refers to entry of the AAV vector or virion into the cell and the transfer of genetic material contained within the AAV vector or virion into the cell to obtain expression from the vector genome. In some cases, but not all cases, transduction and tropism may correlate.

As used herein, the term “reference process” refers to a process or method, e.g., for producing recombinant adeno-associated virus (rAAV) vector-based gene therapies, against which a test process, e.g., a method for hybrid HAVE as described herein, is compared.

### II. Production of Recombinant Adeno-Associated Virus (Raav) Vectors

The development of recombinant adeno-associated virus (rAAV) vectors has been indispensable in the actualization of medically impactful gene therapies. However, manufacture of rAAV vectors tailored for various disease indications at the requisite scale for clinical demand continues to be a rate-limiting step in the progress of gene therapy, and strategically distinguished bioprocesses have been developed to manufacture rAAV at high yield, potency, and purity. Without wishing to be bound by theory, such processes are complicated in the case of rAAV by this species biological need for at minimum four categories of components, including: 1) a cellular environment capable of robustly supporting viral replication, 2) an rAAV genomic template containing a therapeutic transgene flanked by cis-acting AAV DNA elements (e.g., AAV inverted terminal repeats, or “ITRs”), 3) the AAV replication and capsid proteins (e.g., encoded by a rep/cap gene cassette), and 4) complementing “helper” genes provided from any of several identified sources. Various naturally occurring genes encoded by herpes simplex virus 1 have been demonstrated to be minimally sufficient as AAV helper genes or otherwise beneficial towards its replication, and as such, the use of recombinant herpes simplex virus (rHSV) strains as vectors for independently delivering to host production cell both the rep/cap cassette and the therapeutic gene of interest (GOI) to be vectorized has proved auspicious. This process of herpesvirus-assisted vector expansion (HAVE) has been successfully applied to generate material used in clinical trials, with demonstrated safety and efficacy. (See, e.g., U.S. Patent Application Publication No. 20060263883, incorporated by reference in its entirety herein).

However, rHSV vectors are costly to produce, and more importantly, due to often unforeseen interactions between particular GOI's and the herpes simplex virus replication cycle, some genes cannot be stably vectorized within rHSV (including but not limited to overexpressed transmembrane proteins), and thus cannot be vectorized into rAAV via the HAVE process. In some embodiments, the transmembrane protein may be a light-sensing protein useful in optogenetic applications, such as optogenetic gene therapy.

Accordingly, the present disclosure provides novel methods and compositions enabling circumvention of such issues. In some embodiments, the present disclosure provides methods in which an AAV production cell line is co-infected with a seed rAAV vector and an rHSV vector. In some embodiments, the seed rAAV vector may be configured to deliver a GOI-encoding template genome to be encoded into progeny rAAV vectors. In some embodiments, the rHSV vector is configured to deliver both a set of helper genes and a cassette for the expression of rep/cap genes, for example, which determine the capsid serotype (and thus tropism) of progeny rAAV.

Without wishing to be bound by theory, the methods described herein may result in the production of progeny rAAV vectors that may infect additional cells, and serve as a template for additional cycles of rAAV production, thereby providing expansion of rAAV vector by orders of magnitude. The seed rAAV in this process may itself (i) be manufactured by any of many AAV production processes commonly known in the art including but not limited to transfection, herpesvirus-assisted vector expansion (HAVE), or any method described herein, (ii) be manufactured, in theory, at any scale and grade, and (iii) be comprised of any capsid serotype.

The methods described herein, may allow rapid expansion of a rAAV vector from cursory material and conversion of rAAV vector of one serotype into larger quantities of rAAV vector of any other chosen serotypes. Furthermore, the methods described herein may reproducibly achieve yields of rAAV vector in quantities within an order of magnitude of that from production processes commonly known in the art, such as the analogous established HAVE process as described, for example, in U.S. Patent Application Publication No. 20060263883, hereby incorporated by reference in its entirety. In particular embodiments, the methods described herein may be advantageously used where application of the HAVE process is not feasible including but not limited to vectors indicated for treatment of retinal photoreceptor degeneration, autosomal recessive congenital deafness, and/or adrenoleukodystrophy. In other embodiments, the methods described herein may be advantageously used for the amplification of and/or serotype modification of AAV (e.g., recombinant AAV and/or wild-type AAV).

As demonstrated herein, Applicant has successfully executed the methods described herein at exploratory scale (<100 mL shaker cultures) in the production of rAAV vectors encoding either a green fluorescent protein (GFP) expression cassette or a GOI expression cassette (which cannot to-date be vectorized via rHSV), with respective yields of roughly 70,000 DNase-resistant rAAV particles per cell (DRP/cell) and 30,000 DRP/cell, ±2,500 DRP/cell in either case. Characterization of the amount of input rHSV and rAAV vector, specifically, has been performed. Methods of using “refed” rAAV (i.e., use of hybrid HAVE product directly without purification for subsequent production) have also been successfully executed.

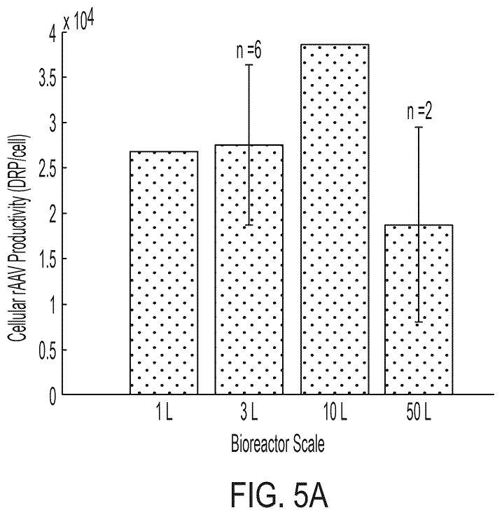

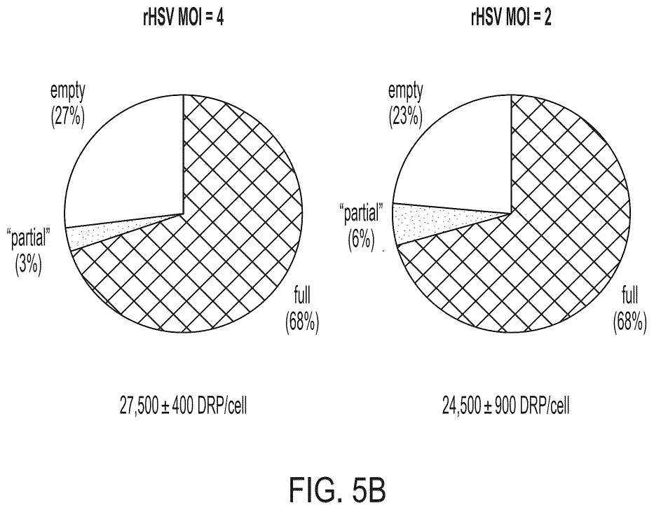

As demonstrated herein, Applicant has successfully executed the methods described herein at variable scales, including, for example, at between about 1 L to about 50 L (shaker cultures) in the production of rAAV vectors encoding either a green fluorescent protein (GFP) expression cassette or a GOI expression cassette (which cannot to-date be vectorized via rHSV), with yields of between about 20,000 DNase-resistant rAAV particles per cell (DRP/cell) to about 40,000 DRP/cell (FIG. 5A). Characterization of the amount of input rHSV and rAAV vector, specifically, has been performed. Methods of using “refed” rAAV (i.e., use of hybrid HAVE product directly without purification for subsequent production) have also been successfully executed.

### Methods of Expansion

The methods as described comprise in certain embodiments co-infecting a mammalian cell capable of growing in suspension with a rHSV vector comprising a nucleic acid sequence encoding an AAV rep and an AAV cap gene each operably linked to a promoter, and a seed rAAV vector comprising a gene of interest, and a promoter operably linked to said gene of interest, flanked by AAV inverted terminal repeats to facilitate packaging of the gene of interest, and allowing the viruses to infect the mammalian cell, thereby producing recombinant AAV viral particles in a mammalian cell.

Any type of mammalian cell that is capable of supporting infection of herpesvirus, with or without, replication of herpesvirus, is suitable for use according to the methods of the invention as described herein. Accordingly, such a mammalian cell can be considered a host cell for the herpesvirus as described in the methods herein. Any cell type for use as a host cell is contemplated by the present disclosure, as long as the cell is capable of supporting infection of herpesvirus. Examples of suitable genetically unmodified mammalian cells include but are not limited to cell lines such as HEK-293 (293), Vero, RD, BHK-21, HT-1080, A549, Cos-7, ARPE-19, and MRC-5. One of skill in the art would be familiar with the wide range of host cells that are available for use in methods for producing an rAAV, in particular examples a rAAV as described in the embodiments herein.

The host cells used in the various embodiments of the present disclosure may be derived, for example, from mammalian cells such as human embryonic kidney cells or primate cells. Other cell types might include, but are not limited to BHK cells or derivatives thereof, Vero cells, CHO cells or any eukaryotic cells for which tissue culture techniques are established. In some embodiments, the cells need not be supportive of “productive infection” (i.e., completing the entire intracellular virus life cycle within the cellular environment), for example, as long as the cells are capable of supporting infection of herpesvirus and delivering gene content for rAAV production. In some embodiments, the cells are herpesvirus permissive. The term “herpesvirus permissive” means that the herpesvirus or herpesvirus vector is able to complete the entire intracellular virus life cycle within the cellular environment. In certain embodiments, methods as described occur in the mammalian cell line BHK, growing in suspension.

The host cell may be derived from an existing cell line, e.g., from a BHK cell line, or developed de novo.

U.S. application No. 20070172846, incorporated by reference in its entirety herein, describes methodologies that have been used to adapt 293 cells into suspension cultures. Graham adapted 293A cells into suspension culture (293N3S cells) by 3 serial passages in nude mice (Graham, J. Gen. Virol., 68 (Pt 3): 937-940, 1987). The suspension 293N3S cells were found to be capable of supporting the replication of El-deleted adenoviral vectors. However, Gamier et al. (Gamier et al., Cytotechnology, 15 (1-3): 145-155, 1994) observed that the 293N35 cells had a relatively long initial lag phase in suspension, a low growth rate, and a strong tendency to clump.

A second method that has been used is a gradual adaptation of 293 A cells into suspension growth (Cold Spring Harbor Laboratories, 293S cells). Gamier et al. (1994) reported the use of 293 S cells for production of recombinant proteins from adenoviral vectors. The authors found that 293S cells were much less clumpy in calcium-free media and a fresh medium exchange at the time of virus infection could significantly increase the protein production. It was found that glucose was the limiting factor in culture without medium exchange.

A recombinant AAV viral particle can also be produced in a mammalian cell by the method comprising co-infecting a mammalian cell capable of growing in suspension with a first recombinant herpesvirus comprising a nucleic acid encoding an AAV rep and an AAV cap gene each operably linked to a promoter; and (ii) a second recombinant herpesvirus comprising a gene of interest, and a promoter operably linked to said gene of interest; and allowing the virus to infect the mammalian cell, and thereby producing recombinant AAV viral particles in a mammalian cell. As described herein, the herpesvirus is a virus selected from the group consisting of: cytomegalovirus (CMV), herpes simplex (HSV) and varicella zoster (VZV) and epstein barr virus (EBV). The recombinant herpesvirus is replication defective. The AAV cap gene has a serotype selected from the group consisting of AAV-1, AAV-2, AAV-3, AAV-4, AAV-5, AAV-6, AAV-7, AAV-8, AAV-9, and rhAAV-10. In certain embodiments, such methods are reference methods, e.g., conventional herpes-assisted vector expansion (HAVE).

Production of Adeno-Associated Virus (rAAV) Vectors by Transfection-Based Processes

U.S. application Ser. Nos. 11/503,775, 11/415,024, and 12/812,671, the entire contents of each of which is incorporated herein by reference in its entirety, describes elements of exemplary rAAV vector production systems. For example, rAAV vector may be produced in vitro by introduction of gene constructs into cells known as producer cells. Known systems for the production of rAAV vector may employ, for example, three fundamental elements: 1) a gene cassette containing a gene of interest, 2) a gene cassette containing AAV rep and cap genes, and 3) a source of “helper” virus proteins.

The first gene cassette is constructed with the gene of interest flanked by inverted terminal repeats (ITRs) from AAV. ITRs function to direct integration of the gene of interest into the host cell genome and are essential for encapsidation of the recombinant genome. (Hermonat and Muzyczka, 1984, Samulski, et al., 1983).

The second gene cassette contains rep and cap AAV genes encoding proteins needed for replication and packaging of rAAV. The rep gene encodes four proteins (e.g., Rep 78, 68, 52 and 40) for DNA replication. The cap genes encode three structural proteins (e.g., VP1, VP2, and VP3) that make up the virus capsid (Muzyczka and Berns, 2001).

The third element is required because AAV does not replicate on its own. Helper functions are protein products from helper DNA viruses that create a cellular environment conducive to efficient replication and packaging of rAAV. Traditionally, adenovirus (Ad) has been used to provide helper functions for rAAV, but herpesviruses can also provide these functions as discussed below.

Production of rAAV vectors for gene therapy is carried out in vitro, using suitable producer cell lines such as BHK cells or derivatives thereof grown in suspension. Other cell lines suitable for use in the invention include HEK-293 (293), Vero, RD, BHK-21, HT-1080, A549, Cos-7, ARPE-19, and MRC-5.

Any cell type can be used as a host cell, as long as the cell is capable of supporting infection of a herpesvirus. One of skill in the art would be familiar with the wide range of host cells that can be used in the production of herpesvirus from host cells. Examples of suitable genetically unmodified mammalian host cells may include, but are not limited to, cell lines such as HEK-293 (293), Vero, RD, BHK-21, HT-1080, A549, Cos-7, ARPE-19, and MRC-5.

In particular embodiments, a host cell is adapted for growth in suspension culture. In certain embodiments of the present disclosure, the host cells are Baby Hamster Kidney (BHK) cells. BHK cell line grown in suspension is derived from an adaptation of the adherent BHK cell line. Both cell lines are available commercially. In certain embodiments, the cells are suspension-adapted BHK cells (sBHK). A well-known strategy for delivering all of the required elements for rAAV production utilizes two plasmids and a helper virus. This method relies on transfection of the producer cells with plasmids containing gene cassettes encoding the necessary gene products, as well as infection of the cells with Ad to provide the helper functions. This system employs plasmids with two different gene cassettes. The first is a proviral plasmid encoding the recombinant DNA to be packaged as rAAV. The second is a plasmid encoding the rep and cap genes. To introduce these various elements into the cells, the cells are infected with Ad as well as transfected with the two plasmids. The gene products provided by Ad are encoded by the genes E1a, E1b, E2a, E4orf6, and Va (Samulski et al., 1998; Hauswirth et al., 2000; Muzyczka and Burns, 2001). Alternatively, in more recent protocols, the Ad infection step can be replaced by transfection with an adenovirus “helper plasmid” containing the VA, E2A and E4 genes (Xiao, et al., 1998, Matsushita, et al., 1998).

While Ad has been used conventionally as the helper virus for rAAV production, it is known that other DNA viruses, such as herpes simplex virus type 1 (HSV-1) can be used as well. The minimal set of HSV-1 genes required for AAV2 replication and packaging has been identified, and includes the early genes UL5, UL8, UL52 and UL29 (Muzyczka and Burns, 2001). These genes encode components of the HSV-1 core replication machinery, i.e., the helicase, primase, primase accessory proteins, and the single-stranded DNA binding protein (Knipe, 1989; Weller, 1991). This rAAV helper property of HSV-1 has been utilized in the design and construction of a recombinant herpes virus vector capable of providing helper virus gene products needed for rAAV production (Conway et al., 1999).

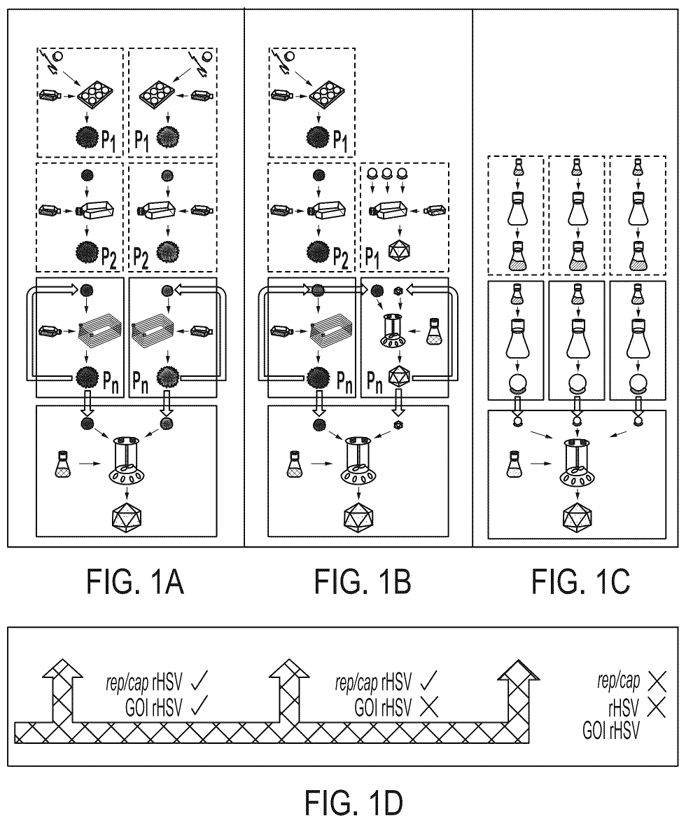

A simplified process flow diagram of an exemplary transfection-based process is shown in FIG. 1C. As shown in FIG. 1C, each of up to three bacterial cultures containing rep/cap-encoding, helper gene-encoding, or GOI-encoding plasmids can be expanded and processed to generate plasmids for use in manufacture of drug product.

Production of Adeno-Associated Virus (rAAV) Vectors by Herpes-Assisted Vector Expansion (HAVE)

U.S. Patent Application Publication No. 20060263883, incorporated by reference in its entirety herein, describes an exemplary process for the production of recombinant adeno-associated virus (rAAV) vectors called herpes-assisted vector expansion (HAVE) in which mammalian cells are co-infected with two replication-defective recombinant herpes simplex virus (rHSV) vectors, each contributing genetic activities which together enable efficient generation of rAAV vector without generation of additional rHSV virions. In this approach, generation of rAAV vector without generation of additional rHSV virions is achieved by deletion of the essential herpes simplex virus gene ICP27 and insertion of either of two classes of transgene cassettes into the locus of the herpes simplex virus thymidine kinase gene, while leaving the remaining roughly 150 kb of rHSV genetic content intact. In some embodiments, for one of these rHSV vectors, the rep/cap rHSV vector, the transgene cassette encodes the adeno-associated virus serotype 2 (AAV2) replication gene rep and the structural gene cap of variable adeno-associated virus serotype, depending on the tropism appropriate to the disease target of therapeutic interest. In some embodiments, for the other rHSV vector, the gene of interest (GOI) rHSV vector, the transgene cassette encodes a therapeutic gene cassette specific to the disease indication to be treated, with this GOI flanked by AAV2 inverted terminal repeats (ITRs), which provides the template genome for rAAV DNA replication and encapsidation. By nature of their battery of wild-type herpes simplex virus genes, each vector contributes helper functions that enable expression and activity of the rep and cap polypeptides.

A generalized process flow diagram of an exemplary herpes-assisted vector expansion (HAVE) platform is shown in FIG. 1A. As shown in FIG. 1A, a rep/cap-encoding rHSV vector and a gene of interest (GOI)-encoding rHSV vector can be propagated in a complementing culture (e.g., with an adherent cell line) to establish banks that are subsequently coinfected into a bioreactor culture (e.g., containing a suspension cell line), to produce rAAV ultimately processed to drug product.

Production of Adeno-Associated Virus (rAAV) Vectors by Hybrid Herpes-Assisted Vector Expansion (HAVE)

Although the HAVE process may be successfully used for the manufacture of drug product for multiple clinical stage programs. The process itself has also undergone extensive development to press its advantages in productivity, scalability, and product quality. For example, one of the process changes which may be adjusted during the manufacture of clinical trial material is the multiplicity of infection (MOI) configuration, defined as the combination of ratios of inoculated virus of each vector class to the viable cells at the time of infection. For example, in preceding version of the HAVE manufacturing process, the MOI configuration was “4:2”; and MOI of 4 for the rep/cap rHSV and 2 for the GOI rHSV. Experiments into the characterization of this parameter lead to the transition of a 2:1 MOI configuration. While the viability of this improved MOI configuration was highly advantageous from a cost-savings standpoint of input rHSV requirements, high cellular rAAV productivity at this MOI was surprising. A basic investigation into the mechanism by which such productivities were attained with limited input rHSV lead to the discovery that nascent rAAV generated during HAVE could itself serve as a GOI vector, delivering template for rAAV DNA replication and encapsidation, and thus converting cells not initially infected with the GOI rHSV into rAAV vector producing cells.

Applicant has discovered that even small quantities of input rAAV can be expanded via coinfection alongside a chosen rep/cap rHSV vector, without requiring the generation, propagation, and deployment of a GOI rHSV. This hybrid variant of the HAVE process was developed by Applicant in a series of small-scale flask-format experiments, ultimately establishing the “hybrid HAVE” process as a bioprocess platform with advantages in flexibility over the conventional HAVE process, albeit with a compromise in rAAV yield over conventional HAVE, while retaining the advantages in yield and scalability over the transfection-based rAAV production processes. In particular, the hybrid HAVE process provided a timely avenue for rapid manufacturing of rAAV vector for those GOI cassettes which have to-date proven to be incompatible with vectorization into rHSV. In particular, for GOI encoding a protein, such as a transmembrane protein, that is incompatible with vectorization with rHSV vectors, the hybrid HAVE process provided a robust, scalable, and highly productive method for manufacturing of rAAV vector. In some embodiments, the transmembrane protein may be a light-sensing protein useful in optogenetic applications, such as optogenetic gene therapy.

A generalized process flow diagram of an exemplary hybrid HAVE process is shown in FIG. 1B. As shown in FIG. 1B, initial GOI-encoding rAAV (“seed rAAV”) can be obtained through various means, for example, by a transfection-based process at small scale. Existing rAAV of any capsid serotype can be expanded via coinfection of cultures (e.g., bioreactors of sub-manufacturing scale) with an rHSV vector encoding a cap gene that determines the desired capsid serotype of the rAAV product bank. Seed rAAV vector can be expanded recursively to arbitrary passage number and scale before the terminal production processed to drug product. FIG. 1D shows a diagrammatic relationship communicating relative productivity and versatility advantages of the hybrid HAVE process compared to transfection-based processes and herpes-assisted vector expansion (HAVE) processes.

In certain embodiments, the instant invention provides production of recombinant AAV viral particles in cells growing in suspension. Suspension or non-anchorage dependent cultures from continuous established cell lines are the most widely used means of large scale production of cells and cell products. Large scale suspension culture based on fermentation technology has clear advantages for the manufacturing of mammalian cell products. The processes are relatively simple to operate and straightforward to scale up. Homogeneous conditions can be provided in the bioreactor which allows for precise monitoring and control of temperature, dissolved oxygen, and pH, and ensure that representative samples of the culture can be taken. The rHSV vectors and/or rAAV vectors used are readily propagated to high titer on permissive cell lines both in tissue culture flasks and bioreactors, and provide a production protocol amenable to scale-up for virus production levels necessary for clinical and market production.

Cell culture in stirred tank bioreactors provides very high volume-specific culture surface area and has been used for the production of viral vaccines (Griffiths, 1986). Furthermore, stirred tank bioreactors have industrially been proven to be scalable. One example is the multiplate CELL CUBE® cell culture system. The ability to produce infectious viral vectors is increasingly important to the pharmaceutical industry, especially in the context of gene therapy.

As used herein, a “bioreactor” refers to any apparatus that can be used for the purpose of culturing cells. Growing cells according to the present disclosure in a bioreactor allows for large scale production of fully biologically-active cells capable of being infected by the rHSV vectors and/or rAAV vectors of the present disclosure.

Bioreactors have been widely used for the production of biological products from both suspension and anchorage dependent animal cell cultures. Most large-scale suspension cultures are operated as batch or fed-batch processes because they are the most straightforward to operate and scale up. However, continuous processes based on chemostat or perfusion principles are available.

The bioreactor system can, in certain embodiments, be set up to include a system to allow for media exchange. For example, filters may be incorporated into the bioreactor system to allow for separation of cells from spent media to facilitate media exchange. In some embodiments of the present methods for producing rHSV vectors and/or rAAV vectors, media exchange and perfusion is conducted beginning on a certain day of cell growth. For example, media exchange and perfusion can begin on day 3 of cell growth. The filter may be external to the bioreactor, or internal to the bioreactor.

### III. Adeno-Associated Virus (Aav)

Adeno-Associated Virus (AAV) is a non-pathogenic single-stranded DNA parvovirus. AAV has a capsid diameter of about 20 nm. Each end of the single-stranded DNA genome contains an inverted terminal repeat (ITR), which is the only cis-acting element required for genome replication and packaging. The AAV genome carries two viral genes: rep and cap. The virus utilizes two promoters and alternative splicing to generate four proteins necessary for replication (Rep78, Rep 68, Rep 52 and Rep 40). A third promoter generates the transcript for three structural viral capsid proteins, 1, 2 and 3 (VP1, VP2 and VP3), through a combination of alternate splicing and alternate translation start codons (Berns K I, Linden R M. The cryptic life style of adeno-associated virus. Bioessays. 1995; 17:237-45). The three capsid proteins share the same C-terminal 533 amino acids, while VP2 and VP1 contain additional N-terminal sequences of 65 and 202 amino acids, respectively. The AAV virion contains a total of 60 copies of VP1, VP2, and VP3 at a 1:1:20 ratio, arranged in a T=1 icosahedral symmetry (Rose J A, Maizel J V Jr, Inman J K, Shatkin A J. Structural proteins of adenovirus-associated viruses. J Virol. 1971; 8:766-70). AAV requires Adenovirus (Ad), Herpes Simplex Virus (HSV) or other viruses as a helper virus to complete its lytic life-cycle (Atchison R W, Casto B C, Hammon W M. Adenovirus-Associated Defective Virus Particles. Science. 1965; 149:754-6; Hoggan M D, Blacklow N R, Rowe W P. Studies of small DNA viruses found in various adenovirus preparations: physical, biological, and immunological characteristics. Proc Natl Acad Sci USA. 1966; 55:1467-74). In the absence of the helper virus, wt AAV establishes latency by integration with the assistance of Rep proteins through the interaction of the ITR with the chromosome (Berns et al., 1995).

### IV. AAV Serotypes

There are a number of different AAV serotypes, including AAV-1, AAV-2, AAV-3, AAV-4, AAV-5, AAV-6, AAV-7, and AAV-8, AAV-9, and rh-AAV-10. In vivo studies have shown that the various AAV serotypes display different tissue or cell tropisms. For example, AAV-1 and AAV-6 are two serotypes that are efficient for the transduction of skeletal muscle (Gao G P, Alvira M R, Wang L, et al. Novel adeno-associated viruses from rhesus monkeys as vectors for human gene therapy. Proc Natl Acad Sci USA. 2002; 99:11854-11859; Xiao W, Chirmule N, Berta S C, et al. Gene therapy vectors based on adeno-associated virus type 1. J Virol. 1999; 73:3994-4003; Chao H, Liu Y, Rabinowitz J, et al. Several log increase in therapeutic transgene delivery by distinct adeno-associated viral serotype vectors. Mol Ther. 2000; 2:619-623). AAV-3 has been shown to be superior for the transduction of megakaryocytes (Handa A, Muramatsu S, Qiu J, Mizukami H, Brown K E. Adeno-associated virus (AAV)-3-based vectors transduce haematopoietic cells not susceptible to transduction with AAV-2-based vectors. J Gen Virol. 2000; 81:2077-2084). AAV-5 and AAV-6 infect apical airway cells efficiently (Zabner J, Seiler M, Walters R, et al. Adeno-associated virus type 5 (AAV5) but not AAV2 binds to the apical surfaces of airway epithelia and facilitates gene transfer. J Virol. 2000; 74:3852-3858; Halbert C L, Allen J M, Miller A D. Adeno-associated virus type 6 (AAV6) vectors mediate efficient transduction of airway epithelial cells in mouse lungs compared to that of AAV2 vectors. J Virol. 2001; 75:6615-6624.). AAV-2, AAV-4, and AAV-5 transduce different types of cells in the central nervous system (Davidson B L, Stein C S, Heth J A, et al. Recombinant adeno-associated virus type 2, 4, and 5 vectors: transduction of variant cell types and regions in the mammalian central nervous system. Proc Natl Acad Sci USA. 2000; 97:3428-3432). AAV-8 and AAV-5 can transduce liver cells better than AAV-2 (Gao G P, Alvira M R, Wang L, et al. Novel adeno-associated viruses from rhesus monkeys as vectors for human gene therapy. Proc Natl Acad Sci USA. 2002; 99:11854-11859; Mingozzi F, Schuttrumpf J, Arruda V R, et al. Improved hepatic gene transfer by using an adeno-associated virus serotype 5 vector. J Virol. 2002; 76:10497-10502). WO99/61601, incorporated by reference in its entirety herein, shows that AAV5 based vectors transduced certain cell types (cultured airway epithelial cells, cultured striated muscle cells and cultured human umbilical vein endothelial cells) at a higher efficiency than AAV2, while both AAV2 and AAV5 showed poor transduction efficiencies for NIH 3T3, skbr3 and t-47D cell lines. AAV-4 was found to transduce rat retina most efficiently, followed by AAV-5 and AAV-1 (Rabinowitz J E, Rolling F, Li C, et al. Cross-packaging of a single adeno-associated virus (AAV) type 2 vector genome into multiple AAV serotypes enables transduction with broad specificity. J Virol. 2002; 76:791-801; Weber M, Rabinowitz J, Provost N, et al. Recombinant adeno-associated virus serotype 4 mediates unique and exclusive long-term transduction of retinal pigmented epithelium in rat, dog, and nonhuman primate after subretinal delivery. Mol Ther. 2003; 7:774-781).

Since the development of naturally occurring AAV serotypes into gene therapy vectors, much effort has been focused towards understanding the tropism of each serotype so that further modification to the virus could be performed to enhance the efficiency of gene transfer. One approach is to swap domains from one serotype capsid to another, and thus create hybrid vectors with desirable qualities from each parent. As the viral capsid is responsible for cellular receptor binding, the understanding of viral capsid domain(s) critical for binding is important. Mutation studies on the viral capsid (mainly on AAV2) performed before the availability of the crystal structure were mostly based on capsid surface functionalization by adsorption of exogenous moieties, insertion of peptide at a random position, or comprehensive mutagenesis at the amino acid level. Choi et al. (Curr Gene Ther. 2005 June; 5 (3): 299-310), incorporated by reference in its entirety herein, describe different approaches and considerations for hybrid serotypes.

The invention includes a method for producing rAAV particles with capsid proteins expressed by multiple serotypes of AAV. This is achieved by co-infection of producer cells with a rHSV expression virus, a rAAV expression virus, and/or with a rHSV-rep2capX helper virus in which the cap gene products are derived from serotypes of AAV other than, or in addition to, AAV2. However, in particular embodiments, the present disclosure provides a method for producing rAAV particles with capsid proteins expressed by multiple serotypes of AAV in which an AAV production cell line is co-infected with a seed rAAV vector and an rHSV vector, the former of which delivers GOI-encoding template genomes to be encoded into progeny rAAV vectors, and the latter of which delivers both a set of helper genes and a cassette for the expression of rep/cap genes, which determine the capsid serotype (and thus tropism) of progeny rAAV. In this process, progeny rAAV vectors may infect additional cells, and serve as template for further cycles of rAAV production, providing expansion of rAAV vector by orders of magnitude. The seed rAAV in this process can itself (i) be manufactured by any of many AAV production processes, including but not limited to transfection, HAVE, or the presently discussed “hybrid” HAVE process, (ii) be manufactured, in theory, at any scale and grade, and (iii) be of comprised of any capsid serotype. The former two of the preceding points allow rapid expansion of vector from cursory material, and the lattermost point allows conversion of rAAV vector of one serotype into larger quantities of rAAV vector of any other chosen serotypes. Recombinant AAV vectors have generally been based on AAV-2 capsids. It has recently been demonstrated that rAAV vectors based on capsids from AAV-1, AAV-3, AAV-4, AAV-5, AAV-8 or AAV-9 serotypes differ from AAV-2 in their tropism.

Capsids from other AAV serotypes offer advantages in certain in vivo applications over rAAV vectors based on the AAV-2 capsid. First, the appropriate use of rAAV vectors with particular serotypes may increase the efficiency of gene delivery in vivo to certain target cells that are poorly infected, or not infected at all, by AAV-2 based vectors. Secondly, it may be advantageous to use rAAV vectors based on other AAV serotypes if re-administration of rAAV vector becomes clinically necessary. It has been demonstrated that re-administration of the same rAAV vector with the same capsid can be ineffective, possibly due to the generation of neutralizing antibodies generated to the vector (Xiao, et al., 1999, Halbert, et al., 1997). This problem may be avoided by administration of a rAAV particle whose capsid is composed of proteins from a different AAV serotype, not affected by the presence of a neutralizing antibody to the first rAAV vector (Xiao, et al., 1999). For the above reasons, recombinant AAV vectors constructed using cap genes from serotypes including and in addition to AAV-2 are desirable. It will be recognized that the construction of recombinant HSV vectors similar to rHSV but encoding the cap genes from other AAV serotypes (e.g. AAV-1, AAV-2, AAV-3, AAV-5 to AAV-9) is achievable using the methods described herein to produce rHSV. In certain preferred embodiments of the invention as described herein, recombinant AAV vectors constructed using cap genes from different AAV are preferred. The significant advantages of construction of these additional rHSV vectors are ease and savings of time, compared with alternative methods used for the large-scale production of rAAV. In particular, the difficult process of constructing new rep and cap inducible cell lines for each different capsid serotypes is avoided.

Accordingly, in some embodiments, the predetermined capsid serotype comprises an AAV serotype selected from the group consisting of AAV-1, AAV-2, AAV-2tYF, AAV-3, AAV-3b, AAV-4, AAV-5, AAV-6, AAV-7, AAV-8, and AAV-9, or variants thereof. In some embodiments, the AAV serotype is AAV-1. In some embodiments, the AAV serotype is AAV-2. In some embodiments, the AAV serotype is AAV-2tYF. In some embodiments, the AAV serotype is AAV-3. In some embodiments, the AAV serotype is AAV-3b. In some embodiments, the AAV serotype is AAV-4. In some embodiments, the AAV serotype is AAV-5. In some embodiments, the AAV serotype is AAV-6. In some embodiments, the AAV serotype is AAV-7. In some embodiments, the AAV serotype is AAV-8. In some embodiments, the AAV serotype is AAV-9. For example, the predetermined capsid serotype may be AAV-2 or AAV-2tYF. In some embodiments, the predetermined capsid serotype is a non-naturally occurring, synthetic or engineered capsid. In some embodiments, such caspids are tropic for ocular or retinal cells, for example AAV2.7m8.

### V. Nucleic Acids

The present disclosure provides promoters, cassettes, vectors, and methods that can be used in the treatment of diseases and disorders associated with a deficiency of a gene of interest (GOI). According to some aspects, the disclosure relates to delivering a heterologous nucleic acid to a subject comprising administering a gene therapy to the subject.

### Making the Nucleic Acids of the Invention

A nucleic acid molecule (including, for example, a GOI nucleic acid) of the present disclosure can be isolated using standard molecular biology techniques. Using all or a portion of a nucleic acid sequence of interest as a hybridization probe, nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

A nucleic acid molecule for use in the methods of the invention can also be isolated by the polymerase chain reaction (PCR) using synthetic oligonucleotide primers designed based upon the sequence of a nucleic acid molecule of interest. A nucleic acid molecule used in the methods of the invention can be amplified using cDNA, mRNA or, alternatively, genomic DNA as a template and appropriate oligonucleotide primers according to standard PCR amplification techniques.

Furthermore, oligonucleotides corresponding to nucleotide sequences of interest can also be chemically synthesized using standard techniques. Numerous methods of chemically synthesizing polydeoxynucleotides are known, including solid-phase synthesis which has been automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein). Automated methods for designing synthetic oligonucleotides are available. See e.g., Hoover, D. M. & Lubowski, 2002. J. Nucleic Acids Research, 30 (10): e43.

Many embodiments of the invention involve a GOI nucleic acid. Some aspects and embodiments of the invention involve other nucleic acids, such as isolated promoters or regulatory elements. A nucleic acid may be, for example, a cDNA or a chemically synthesized nucleic acid. A cDNA can be obtained, for example, by amplification using the polymerase chain reaction (PCR) or by screening an appropriate cDNA library. Alternatively, a nucleic acid may be chemically synthesized.

### Promoters

In some embodiments, the systems and methods disclosed herein can be used with a promoter. As used herein, a “promoter” refers to, for example, a region of DNA that facilitates the transcription of a particular gene, as is well known in the art.

The promoter can be a constitutive, inducible or repressible promoter. In some embodiments, the promoter is a synthetic promoter. In some embodiments, the promoter is highly specific for tissue-specific expression. In some embodiments, the promoter is capable of expressing the heterologous nucleic acid in a cell of a specific tissue and/or organ. In some embodiments, the promoter is capable of expressing the heterologous nucleic acid in a cell of the eye. In some embodiments, the promoter is capable of expressing the heterologous nucleic acid in photoreceptor cells or RPE. In some embodiments, the promoter is capable of expressing the heterologous nucleic acid in a multitude of retinal cells.

In some embodiments, the promoter may be a naturally occurring promoter, e.g., a wild-type promoter. As used herein, the term “naturally occurring” or “wild-type” promoter refers to a promoter that is derived from a naturally occurring source, such as an endogenous gene of interest (GOI) promoter. In other embodiments, the promoter may comprise segments of a GOI, for example, a human GOI gene. In other embodiments, the GOI is a GOI gene from a non-human animal.

In some embodiments, the promoter is capable of promoting expression of a transgene. A “transgene” refers to a segment of DNA containing a gene sequence that has been isolated from one organism and is introduced into a different organism. For example, to treat an individual who has disease or disorder caused by a mutation of a GOI, a wild-type (i.e., non-mutated, or functional variant) GOI may be administered using an appropriate vector. The wild-type gene is referred to as a “transgene.” In preferred embodiments, the transgene is a wild-type version of a GOI. In one such embodiment, the transgene is derived from a human gene.

In some embodiments, the promoter is a native AAV promoter. In some embodiments, the promoter is a constitutive promoter. In some embodiments, the promoter is a cell type-specific promoter.

### Expression Cassettes

In another aspect, the present disclosure provides a transgene expression cassette that includes (a) a promoter; (b) a nucleic acid comprising a gene of interest (GOI) nucleic acid as described herein; and (c) minimal regulatory elements. A promoter of the invention includes the promoters discussed supra.

According to some embodiments, a nucleic acid of the present disclosure encodes a gene of interest (GOI). In some embodiments, the GOI encodes a therapeutic protein. In some embodiments, the GOI is codon-optimized for human expression. In some embodiments, the GOI encodes a therapeutic RNA, such antisense RNA or siRNA or miRNA, or encodes gene editing reagents i.e., guide nucleic acids and nucleases. In some embodiments, the GOI encodes a therapeutic, non-protein-encoding RNA or gene editing reagent. In some embodiments, the GOI is incompatible with vectorization with rHSV vectors (e.g., cannot be inserted into and/or expressed from the viral genome). In some embodiments, and in particular in some embodiments in which the GOI encodes a protein that is incompatible with vectorization with rHSV vectors, the GOI encodes a transmembrane protein. In some embodiments, such a transmembrane protein may be a monotopic, biotopic, or polytopic integral transmembrane protein. The transmembrane protein may include one or more hydrophobic regions, which regions contain α-helical portions. The transmembrane protein may be, for example, a channel protein, such as an ion channel, or a carrier protein, and/or may have one of the following functions: receptor, receptor ligand, structural (e.g., beta-dystroglycan), adhesion, transport (e.g., ABC transporter, P-glycoprotein) or gene regulation. In some embodiments, the transmembrane protein is a G protein-coupled receptor (GPCR) such as a cadherin (calcium-dependent adhesion molecule) or an opsin, or is a G protein-coupled inwardly rectifying potassium channel (GIRK) such as GIRK-1, -2, -3 or -4. In some embodiments the transmembrane protein is a gap junction protein (GJP), such as a connexin (e.g., gap junction beta 2), and/or may have protein kinase activity, e.g., human insulin receptor.

In some embodiments the transmembrane protein may be a light-sensing protein useful in optogenetic applications, such as optogenetic gene therapy. Such a light-sensing protein may be an opsin, such as a microbial or vertebrate opsin (McClements et al (2020), Frontiers in Neuroscience, vol 14, article 570909). Such a microbial opsin may be channelrhodopsin-2 (ChR2) or an engineered variant thereof such as ReaChR or ChrimsonR, halorhodopsin (NpHR) or enhanced halorhodopsin (eNpHR) or Jaws. Such a vertebrate opsin may be rhodopsin (RHO), short-wave cone opsin (SWC), medium-wave cone opsin (MWC), long-wave cone opsin (LWC), melanopsin (OPN4), or an engineered opsin such as Chronos (ChR) or multicharacteristic (polychromatic) opsin (MCO; U.S. Pat. No. 11,180,537).

A “GOI nucleic acid” refers to a nucleic acid that comprises the gene of interest (GOI) or a portion thereof, or a functional variant of the GOI or a portion thereof. A functional variant of a gene includes a variant of the gene with minor variations such as, for example, silent mutations, single nucleotide polymorphisms, missense mutations, and other mutations or deletions that do not significantly alter gene function.

In certain embodiments, the nucleic acid is a human nucleic acid (i.e., a nucleic acid that is derived from a human GOI). In other embodiments, the nucleic acid is a non-human nucleic acid (i.e., a nucleic acid that is derived from a non-human GOI).

According to some embodiments, the recombinant nucleic acid is flanked by at least two ITRs.

“Minimal regulatory elements” are regulatory elements that are necessary for effective expression of a gene in a target cell. Such regulatory elements could include, for example, promoter or enhancer sequences, a polylinker sequence facilitating the insertion of a DNA fragment within a plasmid vector, and sequences responsible for intron splicing and polyadenlyation of mRNA transcripts. In a recent example of a gene therapy treatment for achromatopsia, the expression cassette included the minimal regulatory elements of a polyadenylation site, splicing signal sequences, and AAV inverted terminal repeats. See, e.g., Komaromy et al. The expression cassettes of the invention may also optionally include additional regulatory elements that are not necessary for effective incorporation of a gene into a target cell.

According to some embodiments, the construct comprises a SV (40) polyA.

### Vectors

The present disclosure also provides vectors that include any one of the expression cassettes discussed in the preceding section. In some embodiments, the vector is an oligonucleotide that comprises the sequences of the expression cassette. In specific embodiments, delivery of the oligonucleotide may be accomplished by in vivo electroporation (see, e.g., Chalberg, T W, et al. Investigative Ophthalmology &Visual Science, 46, 2140-2146 (2005) (hereinafter Chalberg et al., 2005)) or electron avalanche transfection (see, e.g., Chalberg, T W, et al. Investigative Ophthalmology &Visual Science, 47, 4083-4090 (2006) (hereinafter Chalberg et al., 2006)). In further embodiments, the vector is a DNA-compacting peptide (see, e.g., Farjo, R, et al. PLOS ONE, 1, e38 (2006) (hereinafter Farjo et al., 2006), where CK30, a peptide containing a cystein residue coupled to polyethylene glycol followed by 30 lysines, was used for gene transfer to photoreceptors), a peptide with cell penetrating properties (see Johnson, L N, et al., Cell-penetrating peptide for enhanced delivery of nucleic acids and drugs to ocular tissues including retina and cornea. Molecular Therapy, 16 (1), 107-114 (2007) (hereinafter Johnson et al., 2007), Barnett, E M, et al. Investigative Ophthalmology & Visual Science, 47, 2589-2595 (2006) (hereinafter Barnett et al., 2006), Cashman, S M, et al. Molecular Therapy, 8, 130-142 (2003) (hereinafter Cashman et al., 2003), Schorderet, D F, et al. Clinical and Experimental Ophthalmology, 33, 628-635 (2005) (hereinafter Schorderet et al., 2005), Kretz, A, et al., Molecular Therapy, 7, 659-669 (2003) (hereinafter Kretz et al. 2003) for examples of peptide delivery to ocular cells), or a DNA-encapsulating lipoplex, polyplex, liposome, or immunoliposome (see e.g., Zhang, Y, et al. Molecular Vision, 9, 465-472 (2003) (hereinafter Zhang et al. 2003), Zhu, C, et al. Investigative Ophthalmology & Visual Science, 43, 3075-3080 (2002) (hereinafter Zhu et al. 2002), Zhu, C., et al. Journal of Gene Medicine, 6, 906-912. (2004) (hereinafter Zhu et al. 2004)).

In preferred embodiments, the vector is a viral vector, such as a vector derived from an adeno-associated virus, an adenovirus, a retrovirus, a lentivirus, a vaccinia/poxvirus, or a herpesvirus (e.g., herpes simplex virus (HSV)). See e.g., Howarth, J L et al., Using viral vectors as gene transfer tools. Cell Biol Toxicol 26:1-10 (2010). In the most preferred embodiments, the vector is an adeno-associated viral (AAV) vector.

Multiple serotypes of adeno-associated virus (AAV), including 12 human serotypes (AAV1, AAV2, AAV3, AAV4, AAV5, AAV6, AAV7, AAV8, AAV9, AAV10, AAV11, and AAV12) and more than 100 serotypes from nonhuman primates have now been identified. Howarth J L et al., 2010. In embodiments of the present disclosure wherein the vector is an AAV vector, the serotype of the inverted terminal repeats (ITRs) of the AAV vector may be selected from any known human or nonhuman AAV serotype. In preferred embodiments, the serotype of the AAV ITRs of the AAV vector is selected from the group consisting of AAV1, AAV2, AAV3, AAV4, AAV5, AAV6, AAV7, AAV8, AAV9, AAV10, AAV11, and AAV12. Moreover, in embodiments of the present disclosure wherein the vector is an AAV vector, the serotype of the capsid sequence of the AAV vector may be selected from any known human or animal AAV serotype. In some embodiments, the serotype of the capsid sequence of the AAV vector is selected from the group consisting of AAV1, AAV2, AAV3, AAV4, AAV5, AAV6, AAV7, AAV8, AAV9, AAV10, AAV11, and AAV12. In preferred embodiments, the serotype of the capsid sequence is AAV2. In some embodiments wherein the vector is an AAV vector, a pseudotyping approach is employed, wherein the genome of one ITR serotype is packaged into a different serotype capsid. See e.g., Zolutuhkin S. et al. Methods 28 (2): 158-67 (2002). In preferred embodiments, the serotype of the AAV ITRs of the AAV vector and the serotype of the capsid sequence of the AAV vector are independently selected from the group consisting of AAV1, AAV2, AAV3, AAV4, AAV5, AAV6, AAV7, AAV8, AAV9, AAV10, AAV11, and AAV12.

Accordingly, in some embodiments, the predetermined capsid serotype comprises an AAV serotype selected from the group consisting of AAV-1, AAV-2, AAV-2tYF, AAV-3, AAV-3b, AAV-4, AAV-5, AAV-6, AAV-7, AAV-8, and AAV-9, or variants thereof. In some embodiments, the AAV serotype is AAV-1. In some embodiments, the AAV serotype is AAV-2. In some embodiments, the AAV serotype is AAV-2tYF. In some embodiments, the AAV serotype is AAV-3. In some embodiments, the AAV serotype is AAV-3b. In some embodiments, the AAV serotype is AAV-4. In some embodiments, the AAV serotype is AAV-5. In some embodiments, the AAV serotype is AAV-6. In some embodiments, the AAV serotype is AAV-7. In some embodiments, the AAV serotype is AAV-8. In some embodiments, the AAV serotype is AAV-9. For example, the predetermined capsid serotype may be AAV-2 or AAV-2tYF. In some embodiments, the predetermined capsid serotype is tropic for ocular cells, such as retinal and/or photoreceptor cells. In some embodiments, the predetermined capsid serotype is a non-naturally occurring, synthetic or engineered capsid. In some embodiments, such caspids are tropic for ocular or retinal cells, for example AAV2.7m8.

In some embodiments of the present disclosure wherein the vector is a rAAV vector, a mutant capsid sequence is employed. Mutant capsid sequences, as well as other techniques such as rational mutagenesis, engineering of targeting peptides, generation of chimeric particles, library and directed evolution approaches, and immune evasion modifications, may be employed in the present disclosure to optimize AAV vectors, for purposes such as achieving immune evasion and enhanced therapeutic output. See e.g., Mitchell A. M. et al. AAV's anatomy: Roadmap for optimizing vectors for translational success. Curr Gene Ther. 10 (5): 319-340.

### Gene of Interest (GOI)

In some embodiments, the GOI encodes a therapeutic agent, a peptide, a polypeptide, a protein, a fusion protein, an oligonucleotide, a DNA molecule, an RNA molecule, an RNAi molecule, a small interfering RNA (siRNA), a short hairpin RNA (shRNA), a microRNA (miRNA), an antisense RNA (asRNA), a gene editing reagent, a guide sequence for a gene editing enzyme (e.g., a guide RNA (gRNA)), a gene editing enzyme (e.g., a nuclease), and/or any combination thereof. In some embodiments, the GOI encodes a therapeutic agent, such as a therapeutic protein. In some embodiments, the GOI encodes a therapeutic agent comprising a membrane protein. In some embodiments, the GOI encodes a therapeutic agent comprising a membrane protein selected from the group consisting of an integral membrane protein, a transmembrane protein, a peripheral membrane protein, a lipid-anchored protein, a portion thereof, and combinations thereof. In some embodiments, the therapeutic agent may be a light-sensing protein useful in optogenetic applications, such as optogenetic gene therapy.

In some embodiments, the GOI is incompatible with vectorization with rHSV vectors (e.g., cannot be inserted into and/or expressed from the viral genome). In some embodiments, and in particular in some embodiments in which the GOI encodes a protein that is incompatible with vectorization with rHSV vectors, the GOI encodes a membrane protein, such as a transmembrane protein. The transmembrane protein may include one or more hydrophobic regions, which regions may contain α-helical portions. The transmembrane protein may be, for example, a channel protein, such as an ion channel, or a carrier protein, and/or may have one of the following functions: receptor, receptor ligand, structural (e.g., beta-dystroglycan), adhesion, transport (e.g., ABC transporter, P-glycoprotein) or gene regulation. In some embodiments, the transmembrane protein is a G protein-coupled receptor (GPCR) such as a cadherin (calcium-dependent adhesion molecule) or an opsin, or is a G protein-coupled inwardly rectifying potassium channel (GIRK) such as GIRK-1, -2, -3 or -4. In some embodiments the transmembrane protein is a gap junction protein (GJP), such as a connexin (e.g., gap junction beta 2), and/or may have protein kinase activity e.g, human insulin receptor. In some embodiments, such a transmembrane protein may be a monotopic, a biotopic, or a polytopic integral transmembrane protein. In some embodiments, the GOI encodes a membrane protein selected from the group consisting of an integral membrane protein, a transmembrane protein, a peripheral membrane protein, a lipid-anchored protein, a portion thereof, and a combination thereof.

In some embodiments the transmembrane protein may be a light-sensing protein useful in optogenetic applications, such as optogenetic gene therapy. Such a light-sensing protein may be an opsin, such as a microbial or vertebrate opsin (McClements et al (2020), Frontiers in Neuroscience, vol 14, article 570909). Such a microbial opsin may be channelrhodopsin-2 (ChR2) or an engineered variant thereof such as ReaChR or ChrimsonR, halorhodopsin (NpHR) or enhanced halorhodopsin (eNpHR) or Jaws. Such a vertebrate opsin may be rhodopsin (RHO), short-wave cone opsin (SWC), medium-wave cone opsin (MWC), long-wave cone opsin (LWC), melanopsin (OPN4), or an engineered opsin such as Chronos (ChR) or multicharacteristic (polychromatic) opsin (MCO; e.g. U.S. Pat. No. 11,180,537).

In some embodiments, the GOI encodes a peptide. In some embodiments, the GOI encodes a polypeptide. In some embodiments, the GOI encodes a protein. In particular embodiments, the GOI encodes a membrane protein. In some embodiments, the GOI encodes an integral membrane protein or a portion thereof, such as an integral monotopic protein or a portion thereof. In some embodiments, the GOI encodes a transmembrane protein or a portion thereof. In some embodiments, the GOI encodes an amino acid sequence of a membrane-spanning protein domain (referred to as a “transmembrane domain”). In some embodiments, the transmembrane protein comprises (i) a single transmembrane α-helix (referred to as a “single-pass” or “bitopic” transmembrane protein); (ii) two or more (e.g., 2, 3, 4, 5, 6, 7, or more) transmembrane α-helices (referred to as a “multi-pass” or “polytopic” transmembrane protein); and/or (iii) a β-sheet comprising multiple (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30 or more) β-strands (referred to as a “β-barrel” transmembrane protein). In some embodiments, the GOI encodes a therapeutic agent comprising a peripheral membrane protein or portion thereof. In some embodiments, the GOI encodes a therapeutic agent comprising a lipid-anchored protein or a portion thereof. In some embodiments, the GOI encodes an amino acid sequence comprising (i) a transmembrane α-helix; (ii) a β-sheet; (iii) a β-barrel; (iv) an amphipathic α-helix; (v) a hydrophobic loop; (vi) a feature capable of covalently binding to a membrane lipid; and/or (vii) a feature capable of forming an electrostatic and/or ionic interaction with a membrane lipid.

In some embodiments, the GOI encodes a fusion protein. In some embodiments, the fusion protein comprises a membrane protein or a portion thereof. In some embodiments, the fusion protein comprises a channelrhodopsin (ChR) or a portion thereof. In some embodiments, the fusion protein comprises a green fluorescent protein (GFP). In certain embodiments, the fusion protein comprises a channelrhodopsin (ChR) or a portion thereof and a green fluorescent protein (GFP). In some embodiments, the fusion protein comprises an amino acid sequence that increases the stability and/or expression of the fusion protein.

In some embodiments, the GOI encodes an amino acid sequence of between about 2 and about 650 amino acids (e.g., about 2, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, about 500, about 505, about 510, about 515, about 520, about 525, about 530, about 535, about 540, about 545, about 550, about 555, about 560, about 565, about 570, about 575, about 580, about 585, about 590, about 595, about 600, about 605, about 610, about 615, about 620, about 625, about 630, about 635, about 640, about 645, or about 650 amino acids). In some embodiments, the GOI encodes an amino acid sequence of between about 100 and about 300 amino acids. In some embodiments, the GOI encodes an amino acid sequence of between about 200 and about 400 amino acids. In some embodiments, the GOI encodes an amino acid sequence of between about 300 and about 500 amino acids. In some embodiments, the GOI encodes an amino acid sequence of between about 400 and about 600 amino acids. In some embodiments, the GOI encodes an amino acid sequence of about 650 or more amino acids.

In some embodiments, the nucleic acid sequence encoding the GOI is codon optimized, e.g., for human expression.

### VI. AAV and Gene Therapy

Gene therapy refers to treatment of inherited or acquired diseases by replacing, altering, or supplementing a gene responsible for the disease. It is achieved by introduction of a corrective gene or genes into a host cell, generally by means of a vehicle or vector. Gene therapy using rAAV holds great promise for the treatment of many diseases. The invention provides a novel method of producing recombinant adeno-associated virus (rAAV), and in particular producing recombinant AAV encoding genes that cannot be stably vectorized within rHSV (e.g., including but not limited to transmembrane proteins), and thus cannot otherwise be vectorized into rAAV via the HAVE process, to support clinical applications. In some embodiments, the transmembrane protein may be a light-sensing protein useful in optogenetic applications, such as optogenetic gene therapy.

To date more than 500 gene therapy clinical trials have been conducted worldwide. Efforts to use rAAV as a vehicle for gene therapy hold promise for its applicability as a treatment for human diseases. Already, some success has been achieved pre-clinically and clinically, using recombinant AAV (rAAV) for the delivery and long-term expression of introduced genes into cells in animals, including clinically important non-dividing cells of the brain, liver, skeletal muscle and lung. In some tissues, AAV vectors have been shown to integrate into the genome of the target cell (Hirata et al. 2000, J. of Virology 74:4612-4620).

An additional advantage of rAAV is its ability to perform this function in non-dividing cell types including hepatocytes, neurons and skeletal myocytes. rAAV has been used successfully as a gene therapy vehicle to enable expression of erythropoietin in skeletal muscle of mice (Kessler et al., 1996), tyrosine hydroxylase and aromatic amino acid decarboxylase in the CNS in monkey models of Parkinson disease (Kaplitt et al., 1994) and Factor IX in skeletal muscle and liver in animal models of hemophilia. At the clinical level, the rAAV vector has been used in human clinical trials to deliver the CFTR gene to cystic fibrosis patients and the Factor IX gene to hemophilia patients (Flotte, et al., 1998, Wagner et al, 1998). Further, AAV is a helper-dependent DNA parvovirus, which is not associated with disease in humans or mammals (Berns and Bohensky, 1987, Advances in Virus Research, Academic Press Inc, 32:243-307). Accordingly, one of the most important attributes of AAV vectors is their safety profile in phase I clinical trials.

AAV gene therapy has been carried out in a number of different pathological settings and to treat various diseases and disorders. For example, in a phase I study, administration of an AAV2-FIX vector into the skeletal muscle of eight hemophilia B subjects proved safe and achieved local gene transfer and Factor IX expression for at least 10 months after vector injection (Jiang et al, Mol Ther. 2006 September; 14 (3): 452-5. Epub 2006 Jul. 5), a phase I trial of intramuscular injection of a recombinant adeno-associated virus alpha 1-antitrypsin (rAAV2-CB-hAAT) gene vector to AAT-deficient adults has been described previously (Flotte et al., Hum Gene Ther. 2004 January; 15 (1): 93-128), and in another clinical trial AAV-GAD gene therapy of the subthalamic nucleus has been shown to be safe and well tolerated by patients with advanced Parkinson's disease (Kaplitt et al. Lancet. 2007 Jun. 23; 369 (9579): 2097-105).

Conventional AAV production methodologies make use of procedures known to limit the number of rAAV that a single producer cell can make. The first of these is transfection using plasmids for delivery of DNA to the cells. It is well known that plasmid transfection is an inherently inefficient process requiring high genome copies and therefore large amounts of DNA (Hauswirth et al., 2000).

Advances toward achieving the desired goal of scalable production systems that can yield large quantities of clinical grade rAAV vectors have largely been made in production systems that utilize transfection as a means of delivering the genetic elements needed for rAAV production in a cell. For example, removal of contaminating adenovirus helper has been circumvented by replacing adenovirus infection with plasmid transfection in a three-plasmid transfection system in which a third plasmid comprises nucleic acid sequences encoding adenovirus helper proteins (Xiao et al. 1998). Improvements in two-plasmid transfection systems have also simplified the production process and increased rAAV vector production efficiency (Grimm et al., 1998). Despite these advances, it is generally recognized that transfection systems are limited in their efficiency by the uptake of exogenous DNA, and in their commercial utility due to scaling difficulties.

Several strategies for improving yields of rAAV from cultured mammalian cells are based on the development of specialized producer cells created by genetic engineering. In one approach, production of rAAV on a large scale has been accomplished by using genetically engineered “proviral” cell lines in which an inserted AAV genome can be “rescued” by infecting the cell with helper adenovirus or HSV. Proviral cell lines can be rescued by simple adenovirus infection, offering increased efficiency relative to transfection protocols. However, as with the earlier transfection methods, adenovirus is introduced into the system that must later be removed. Additionally, the rAAV yield is generally low in proviral cell lines (Qiao et al. 2002a).

There are several further disadvantages that limit approaches using proviral cell lines. The cell cloning and selection process itself can be laborious; additionally, this process must be carried out to generate a unique cell line for each therapeutic gene of interest (GOI). Furthermore, cell clones having inserts of unpredictable stability can be generated from proviral cell lines.

A second cell-based approach to improving yields of rAAV from cells involves the use of genetically engineered “packaging” cell lines that harbor in their genomes either the AAV rep and cap genes, or both the rep-cap and the ITR-gene of interest (Qiao et al., 2002b). In the former approach, in order to produce rAAV, a packaging cell line is either infected or transfected with helper functions, and with the AAV ITR-GOI elements. The latter approach entails infection or transfection of the cells with only the helper functions. Typically, rAAV production using a packaging cell line is initiated by infecting the cells with wild-type adenovirus, or recombinant adenovirus. Because the packaging cells comprise the rep and cap genes, it is not necessary to supply these elements exogenously.

While rAAV yields from packaging cell lines have been shown to be higher than those obtained by proviral cell line rescue or transfection protocols, packaging cell lines typically suffer from recombination events, such as recombination of E1a-deleted adenovirus vector with host 293 cell DNA. Infection with recombinant adenovirus therefore initiates both rAAV production and generation of replication-competent adenovirus. Furthermore, only limited success has been achieved in creating packaging cell lines with stable genetic inserts.

Recent progress in improving yields of rAAV has also been made using approaches based on delivery of helper functions from herpes simplex virus (HSV) using recombinant HSV amplicon systems. Although modest levels of rAAV vector yield, of the order of 150-500 viral genomes (vg) per cell, were initially reported (Conway et al., 1997), more recent improvements in rHSV amplicon-based systems have provided substantially higher yields of rAAV v.g. and infectious particles (ip) per cell (Feudner et al., 2002). Amplicon systems are inherently replication-deficient; however the use of a “gutted” vector, replication-competent (rcHSV), or replication-deficient rHSV still introduces immunogenic HSV components into rAAV production systems. Therefore, appropriate assays for these components and corresponding purification protocols for their removal must be implemented. Additionally, amplicon stocks are difficult to generate in high titer, and often contain substantial parental virus contamination.

It is apparent from the foregoing that there is a clear need for improved large-scale methods for production of high titer, rAAV to overcome the major barrier to the routine use of rAAV for gene therapy.

Moreover, rHSV vectors themselves are costly to produce, and more importantly, due to often unforeseen interactions between particular GOI's and the herpes simplex virus replication cycle, some genes cannot be stably vectorized within rHSV (including but not limited to overexpressed transmembrane proteins, such as those transmembrane proteins discussed above), and thus cannot be vectorized into rAAV via the Herpes-Assisted Vector Expansion (HAVE) process. In some embodiments, the transmembrane protein may be a light-sensing protein useful in optogenetic applications, such as optogenetic gene therapy. The present disclosure provides methods enabling circumvention of such issues and for producing clinically relevant recombinant rAAV vectors, in which an AAV production cell line is co-infected with a seed rAAV vector and an rHSV vector, the former of which delivers GOI-encoding template genomes to be encoded into progeny rAAV vectors, and the latter of which delivers both a set of helper genes and a cassette for the expression of rep/cap genes, which determine the capsid serotype (and thus tropism) of progeny rAAV. In this process, progeny rAAV vectors may infect additional cells, and serve as template for further cycles of rAAV production, providing expansion of rAAV vector by orders of magnitude. The seed rAAV in this process can itself (i) be manufactured by any of many AAV production processes, including but not limited to transfection, HAVE, or the presently discussed “hybrid” HAVE process, (ii) be manufactured, in theory, at any scale and grade, and (iii) be of comprised of any capsid serotype. The former two of the preceding points allow rapid expansion of vector from cursory material, and the lattermost point allows conversion of rAAV vector of one serotype into larger quantities of rAAV vector of any other chosen serotypes. Furthermore, the process reproducibly achieves yields of vector in quantities within an order of magnitude of that from the analogous established HAVE process.

### Diseases to be Treated

In embodiments of the instant invention where the method for producing recombinant AAV viral particles in a mammalian cell comprises co-infecting a mammalian cell capable of growing in suspension with an rHSV vector and a seed rAAV vector comprising a gene of interest, the invention contemplates use of any gene that has therapeutic or potential therapeutic value in the treatment of a disease or genetic disorder. One of skill in the art would be familiar with the wide range of such genes that have been identified.

In certain embodiments, the therapeutic genes involved may be those that encode proteins, structural or enzymatic RNAs, inhibitory products such as antisense RNA or DNA, or any other gene product. In particular, the encoded protein in some embodiments is a transmembrane protein such as one of those discussed above. In some embodiments, the transmembrane protein may be a light-sensing protein useful in optogenetic applications, such as optogenetic gene therapy. Expression is the generation of such a gene product or the resultant effects of the generation of such a gene product. Thus, enhanced expression includes the greater production of any therapeutic gene product or the augmentation of that product's role in determining the condition of the cell, tissue, organ, or organism.

In certain embodiments, the therapeutic gene may encode one or more proteins indicated for treatment of an ocular disease or disorder. In certain embodiments, the therapeutic gene may encode one or more proteins indicated for treatment of retinal photoreceptor degeneration. In certain embodiments, the therapeutic gene may encode one or more proteins indicated for treatment of X-linked Retinoschisis (XLRS). XLRS is an inherited retinal disease caused by mutations in the RS1 gene, which encodes the retinoschisin protein. It is characterized by abnormal splitting of the layers of the retina, resulting in poor visual acuity in young boys, which can progress to legal blindness in adult men. In certain embodiments, the therapeutic gene may comprise a RS1 gene.

In certain embodiments, the therapeutic gene may encode one or more proteins indicated for treatment of achromatopsia (ACHM). ACHM is an inherited retinal disease, which is present from birth and is characterized by the lack of cone photoreceptor function. The condition results in markedly reduced visual acuity, extreme light sensitivity causing day blindness, and complete loss of color discrimination. Best-corrected visual acuity in persons affected by ACHM, even under subdued light conditions, is usually about 20/200, a level at which people are considered legally blind. In certain embodiments, ACHM may be caused by mutations in the Cyclic Nucleotide Gated Channel Subunit Beta 3 (CNGB3) gene and/or the Cyclic Nucleotide Gated Channel Subunit Alpha 3 (CNGA3) gene. In certain embodiments, the therapeutic gene may comprise a CNGB3 gene and/or a CNGA3 gene.

In certain embodiments, the therapeutic gene may encode one or more proteins indicated for treatment of X-linked Retinitis Pigmentosa (XLRP). XLRP is an inherited condition that causes boys to develop night blindness by the time they are ten and progresses to legal blindness by their early forties. In certain embodiments, XLRP may be caused by mutations in the retinitis pigmentosa GTPase regulator (RPGR) gene. In certain embodiments, the therapeutic gene may comprise a RPGR gene.

In certain embodiments, the therapeutic gene may encode one or more proteins indicated for treatment of age-related macular degeneration (AMD). AMD is a retinal disease that usually affects older adults and results in a loss of vision in the center of the visual field (the macula). It is a major cause of blindness and visual impairment in older adults and occurs in “dry” and neovascular, or “wet,” forms. Individuals with mutations in CFH, which is a component of the dysregulated complement pathway, have an increased risk of developing AMD that is six times greater than those who do not have such mutation (Sepp et al., 2006). In certain embodiments, the therapeutic gene may comprise a Complement Factor H (CFH) gene.

In certain embodiments, the therapeutic gene may encode one or more proteins indicated for treatment of advanced retinal disease, including for the treatment of individuals having retinitis pigmentosa (RP) who have lost light sensitivity. RP is a large group of inherited retinal disorders in which progressive degeneration of photoreceptors or retinal pigment epithelium (RPE) leads to vision loss that is independent of a patients' genetic mutation. In Europe and the United States, about 200,000 patients suffer from RP and every year between 15,000 and 20,000 patients with RP suffer vision loss. The clinical manifestations of affected individuals present first as defective dark adaptation or “night blindness,” followed by reduction of peripheral visual fields and, eventually, loss of central vision. While the photoreceptor cell layers of these patients degenerate, the ganglion cell layer remains intact and functional. In certain embodiments, the therapeutic gene may comprise a channelrhodopsin (ChR) protein. In certain embodiments, the therapeutic gene may comprise an opsin protein, such as a channelrhodopsin.

In certain embodiments, the therapeutic gene may encode one or more proteins indicated for amyotrophic lateral sclerosis (ALS, Lou Gehrig's disease) which is an autosomal dominant, fatal adult-onset disease with no truly effective therapy. It is the most common adult-onset motor neuron disease, with approximately ˜75,000 cases in the United States and European Union, and is characterized by upper and lower motor neuron degeneration. Early symptoms are muscle weakness that progresses and ultimately results in respiratory failure, with death usually occurring within 3 to 5 years of diagnosis. There are both sporadic (˜90%) and familial (˜10%) forms of the disease, with the most common genetic cause linked to the C9orf72 gene, representing 30-40% of cases. C9orf72 mutations are also present in frontotemporal dementia (FTD). In certain embodiments, the therapeutic gene may comprise a C9orf72 gene.

In certain embodiments, the therapeutic gene may encode one or more proteins indicated for otology. In certain embodiments, the therapeutic gene may encode one or more proteins indicated for treatment of hearing loss and/or deafness, such as autosomal recessive congenital deafness. Mutations in the gap junction protein beta 2 gene (GJB2) account for approximately 30% of all genetic hearing loss cases, representing approximately 90,000 cases in the United States and European Union markets. Patients with this mutation can have severe-to-profound deafness in both ears that is identified in screening tests routinely performed in newborns. In certain embodiments, the therapeutic gene may comprise a GJB2 gene.

In certain embodiments, the therapeutic gene may encode one or more proteins indicated for treatment of adrenoleukodystrophy (ALD). ALD is an X-linked genetic disorder that causes damage to the nervous system and adrenal glands, primarily in young boys. The cerebral form of ALD in children is characterized by difficulty in school, hearing and vision loss, difficulty swallowing and declining motor control, and progresses rapidly to total disability or death in as little as two years. A second form of ALD is characterized by weakness of the legs that begins in adulthood and progresses to lower limb paralysis. Mutations in the ABCD1 gene cause X-linked adrenoleukodystrophy. The ABCD1 gene provides instructions for producing the adrenoleukodystrophy protein (ALDP), which is involved in transporting certain fat molecules called very long-chain fatty acids (VLCFAs) into peroxisomes. ABCD1 gene mutations result in a shortage (deficiency) of ALDP. When this protein is lacking, the transport and subsequent breakdown of VLCFAs is disrupted, causing abnormally high levels of these fats in the body, which can lead to the breakdown of the protective myelin sheath on nerves in the brain and spinal cord. In certain embodiments, the therapeutic gene may comprise an ABCD1 gene.

In certain embodiments, the therapeutic gene may encode one or more proteins indicated for treatment of frontotemporal dementia (FTD). FTD is a degenerative brain disorder, second only to Alzheimer's disease in terms of prevalence and incidence in the dementia spectrum, is on the rise due to the aging population, and has no approved treatments. Mutations in the progranulin (PGRN) gene are one of the three main genetic causes of FTD, representing approximately 20% of familial FTD, and accounting for 7,500 to 15,000 cases in the United States and European Union. In certain embodiments, the therapeutic gene may comprise a progranulin (PGRN) gene.

### EXAMPLES

It should be appreciated that the invention should not be construed to be limited to the examples that are now described; rather, the invention should be construed to include any and all applications provided herein and all equivalent variations within the skill of the ordinary artisan.

### Example 1. Characterization of the Hybrid HAVE Process

### Methods

### Suspension Cell Culture

The rAAV production process described employs banks of serum-free medium-adapted suspension baby hamster kidney (sBHK) cells, which have been developed in-house for high rAAV productivity when used in HAVE. Culture medium is composed of PF-CHO LS (HyClone SH30359.02) supplemented to 10 mM L-glutamine (Invitrogen 25030-081). To initiate seed trains, vials containing 107 viable cells (vc) in 1 mL of CryoStor CS10 (BioLife Solutions 210102) were thawed from liquid nitrogen at 37° C., diluted to 10 mL with warm culture medium, centrifuged at 100×g for 5 minutes, and diluted to 10 mL in fresh culture medium before seeding into a 125 mL disposable Erlenmeyer culture flask (Corning 431143). Passaging of cultures is done via incubation with shaking agitation set to 110 RPM, at 37° C., with 5.0% CO₂ until a concentration of 1.0-2.0×10⁶ vc/mL is reached, after which a portion of the culture is directly added to fresh culture medium to seed a new culture of the desired volume at a cell concentration of 1.0-5.0×10⁵ vc/mL. Cells exhibit a 12±2 h doubling time during routine culture.

rHSV Vectors

The rHSV vector stocks are propagated in the V27 cell line, a transgenic derivative of the Vero African green monkey kidney cell line, into which the herpes simplex virus 1 ICP27 gene has been integrated (Rice and Knipe, 1990). V27 cells are cultured in DMEM (HyClone SH30284) supplemented to 9.1% with FBS (HyClone SH30071) in T-flasks or CellSTACKs, at 37° C., with 5.0% CO₂. To generate the rep/cap and GOI rHSV vectors used in this program, V27 cells were cotransfected with purified genomic DNA of a recombinant ICP27-deficient herpes simplex virus 1 mutant (Conway et al, 1999) alongside a recombination plasmid encoding, for the case of rep/cap rHSV vectors, the adeno-associated virus serotype 2 (AAV2) rep gene and a recombinant cap gene encoding structural proteins for the indicated capsid genotypes, or for the case of GOI rHSV vectors, a promoter-ORF-polyA cassette immediately flanked by AAV2 ITRs. In the case of the positive production control (PPC) rHSV vector used in the majority of experiments described here, rHSV-CBA-hGFP, this cassette was comprised of a recombination plasmid encoding constitutively-expressed GFP, derived from the plasmid pTR-UF11 (Burger at al., 2004). Nascent recombinants generated via cotransfection were plaque-purified to establish a genetically pure passage 1 (P1) strain of the vector. P1 rHSV stocks were expanded via inoculation of V27 cells in vessels of increasing surface area at a multiplicity of infection (MOI) of 0.01, culture of infected cells at 35° C. with 5.0% CO₂ for 96 hours, harvest via incubation for an additional 30-90 minutes with added 5 M NaCl (Lonza 51202) at a volume of 12.5% the culture volume, clarification by centrifugation at 500×g (flask-format productions) or hollow fiber membrane tangential flow filtration (multi-layer productions), formulation with 5% glycerol (Thermo Scientific AAJ16374), and cryostorage at −80° C. Stocks are titered via plaque assay on V27 cells.

Seed rAAV Vectors

Initial P1 rAAV vector used in hybrid HAVE experiments was, unless otherwise stated, generated by a triple transfection approach, entailing polyethylenimine-mediated (PEI-mediated) cotransfection of three plasmids into adherent HEK293 cells, followed by cell lysis via freeze-thaw, purification via iodixanol gradient ultracentrifugation, diafiltration and concentration via the Amicon Ultra-15 centrifugal device, and final formulation in Balanced Salt Solution containing 0.014% of Tween-20 (BSST), similar to published methods (Zolotukhin et al, 2006) (Reed, Staley, Mayginnes, Pintel, and Tullis, 2006). In the case of the rAAV2tYF-CBA-hGFP seed vector, the plasmids include (i) pTR-UF11, including a green fluorescent protein ORF codon-optimized for human expression (hGFP) driven by the chicken beta-actin (CBA) promoter and terminated by the SV40 polyadenylation signal, as well as a neomycin resistance ORF driven by a fusion promoter derived from the polyomavirus B enhancer from strain F441 and the human herpesvirus 1 thymidine kinase gene and terminated by the bovine growth hormone polyadenylation signal, with both cassettes flanked by AAV2 cis elements mediating DNA replication and encapsidation, (ii) a plasmid encoding the rep2/cap2tYF cassette, pACG-2tYF, and (iii) the adenovirus helper plasmid pALD-X80 (Aldevron).

Flask-Format rAAV Production

Small-scale rAAV productions and evaluations were executed as follows, unless otherwise stated. sBHK cell culture seed trains of arbitrary post-thaw passage number were resuspended in fresh culture medium following centrifugation at 140-280×g for 5 minutes, and subsequently serially infected at a cell density of 1-2×106 vc/mL using the indicated MOI configuration of the indicated vectors (PPC rep/cap rHSV vector rHSV-rep2cap2tYF and PPC GOI rHSV vector rHSV-CBA-hGFP for conventional HAVE productions, if not otherwise stated, whereas the PPC GOI rAAV vector comprised rAAV2tYF-CBA-hGFP). Infected cultures were maintained for an additional 24 h, after which cells were lysed and non-encapsidated DNA was digested via addition of octyl phenol ethoxylate (OPE, HyClone RR11909.01) to a final concentration of 0.9%, addition of MgCl2 (Invitrogen AM9530G) to a final concentration of 2 mM, and Benzonase (EMD Millipore 1016970001) to a final concentration of 25 U/mL, followed by incubation for 2 hours under standard culture conditions. Lysates were then combined with 5 M NaCl (Lonza 51202) at a volume equal to 10% that of the original culture volume, after which lysates were centrifuged at 640 rcf for 7 minutes to remove macroscopic cell debris. Aliquots were prepared from the supernatant, and product was titered via the DRP qPCR assay described below.

rAAV Vector Titration

Concentrations of rAAV vector, in units of DNase-resistant particles (DRP) per milliliter, were determined via a test method in which test articles are incubated firstly with 50 U/mL RQ1 DNase I (Promega M6101) to digest non-encapsidated DNA, secondly with 10 mg/mL proteinase K (Ambion AM2546) to digest capsids and DNase, and thirdly at 95° C. to inactivate enzymes, before released rAAV genomes are quantified via a TaqMan probe qPCR reaction (Life Technologies 4318157) using a qPCR assay targeted to the simian vacuolating virus 40 (SV40) polyadenylation signal within the GOI cassette (sequences below) against a standard curve composed of six 10-fold serial dilutions of an internally validated control spanning 2.0×10¹⁰ to 2.0×10⁵ copies/mL.

T-SV40-F:
5′ AGCAATAGCATCACAAATTTCACAA 3′
T-SV40-R:
5′ CCAGACATGATAAGATACATTGATGAGTT 3′
T-SV40-Pr:
5′ 6-FAM-AGCATTTTTTTCACTGCATTCTAGTTGTGGTTTGTC-
TAMRA 3′

Conventional and Hybrid HAVE Bioreactor-Format rAAV Production

One vial of sBHK cells per bioreactor was thawed and passaged as described above, passaging over five days in flasks of increasing volume. Cultures were seeded into bioreactors of various sizes as described herein (e.g., a 3 L Mobius Single-Use Bioreactor) to a concentration of about 5.0×10⁵ vc/mL in PF CHO LS supplemented to about 10 mM L-glutamine, about 0.16% poloxamer 188, and either about 8.5 ppm Antifoam C for process development runs, or about 1.27 ppm Antifoam ADCF for those runs in the expansion series. The bioreactors were maintained using the DASGIP Parallel Bioreactor System at 280 RPM agitation, about 37° C., about pH 7.2 maintained via 1 M NaOH or sparged CO₂, and about 50% DO with 0.02-0.09 vvm gas flow rate. At about 24 hours post-seeding (hps), bioreactors were fed with a solution containing a volume about 15% that of the working culture volume of Cell Boost 6 supplement and a volume about 0.694% that of the working culture volume of a about 45% glucose solution. At about hps, bioreactors were fed again, using volumes about 10% and about 0.694% of the working culture volume for each of the aforementioned supplements, and the DO was decreased to about 25%. Around about 48 hps, a cell count was taken, and the cultures were concurrently infected at an MOI of about 2 (unless otherwise stated) with the indicated rep/cap-encoding rHSV (rHSV-rep2capSV6d, encoding the cap gene of a novel AAV2-derived capsid, or rHSV-rep2cap2tYF for the process comparison experiment) and either an MOI of 1 with the indicated GOI-encoding rHSV (also referred to as “GOI-encoding rAAV seed vector”) (rHSV-CBA-GJP, or rHSV-GRK1-RPGR or rHSV-CBA-hGFP for the process comparison experiment) for conventional HAVE or an MOI of about 100 with rAAV2tYF-CBA-GFP or rAAV2tYF-GRK1-RPGR for hybrid HAVE. Around about 75 hps, cells were lysed and non-encapsidated DNA was digested via addition of detergent (OPE for all bioreactors except for those used in head-to-head comparison of the conventional HAVE, hybrid HAVE, and transfection processes, which used Tween-20) to a final concentration of about 0.9 to about 1.0%, addition of MgCl₂ to a final concentration of about 2 mM, and Benzonase to a final concentration of >25 U/mL, followed by incubation for about 2-3 hours, after which about 5 M NaCl is added to achieve a final concentration of about 0.5 M.

Variable-Process Platform Bioreactor-Format rAAV Production

In the experiment comparing rAAV production processes, all productions were performed as described above, using 3 L Mobius Single-Use Bioreactors, with the differences described below. For the hybrid HAVE process, the infection step was performed using rHSV-rep2cap2tYF at an MOI of about 2 and rAAV2tYF-GRK1-RPGR at an MOI of about 100 as the respective rep/cap and GOI vectors. For the conventional HAVE process, the infection step was performed using rHSV-rep2cap2tYF at an MOI of about 2 and rHSV-GRK1-RPGR at an MOI of about 1 as the respective rep/cap and GOI vectors. For the transfection process, Expi293F cells were cultured and expanded in Expi293 Expression Medium (supplemented with antifoam), using the manufacturer's recommendations, instead of sBHK-SF cells in PF CHO LS, and at around 24 hps, the cultures were transfected at about 2.0×106 vc/mL with 1 μg per 106 viable cells of plasmid DNA mixture complexed with Fecto VIR-AAV (1 μL per 1 μg DNA, using the manufacturer's recommendations for complexation and delivery. The plasmid mixture comprised the GOI plasmid pTR-GRK1-RPGR, the rep2/cap2tYF plasmid pACG2-2tYF, and the adenovirus 5 helper plasmid pALD-X80, at a molar ratio of 2:2:1. Transfected cells were lysed at about 48 hours post-transfection, rather than 27 hours post-infection. For all processes, cells were lysed via addition of Tween-20 to a final concentration of about 1.0% rather than OPE at around 0.9%.

### Analytical Ultracentrifugation

Proportions of rAAV vectors encapsidated with full length genomes, with sub-genomic-length DNA, and without DNA for provided test articles were assessed by analytical ultracentrifugation.

### Results

### Demonstration of the Hybrid HAVE Process in a Small-Scale Test Model

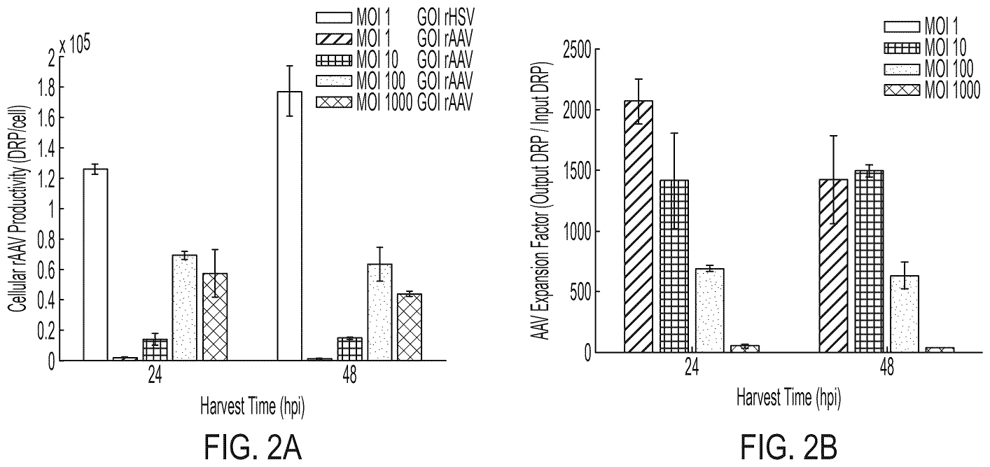

Flask-format cultures of suspension BHK cells at a volume of 40 mL were infected with the rHSV-rep2cap2tYF vector at an MOI of 4 and either the rHSV-CBA-hGFP vector at an MOI of 1 (conventional HAVE) or the rAAV2tYF-CBA-hGFP vector at MOIs spanning a 4-log range (hybrid HAVE). The following day, cultures were lysed via detergent and centrifuged to remove large cell debris, and rAAV in the lysate was quantified via a qPCR assay for DNase-resistant particles (DRP). FIG. 2A shows a bar graph of cellular rAAV productivity (DNase-resistant particles (DRP)/cell) with respect to multiplicity of infection (MOI) at 24-hours and 48-hours harvest time (hpi). Error bar lengths represent twice the standard deviation from the mean for duplicate biological replicates. FIG. 2B shows a bar graph of AAV Expansion Factor (output DRP/input DRP) with respect to multiplicity of infection (MOI) at 24-hours and 48-hours harvest time (hpi). Results are shown in the ratio of input rAAV vector to output rAAV vector to emphasize that even at low productivity conditions, e.g., MOI configurations that would be suboptimal for manufacturing, small quantities of rAAV in sample material can be rapidly amplified.

Demonstration of the rHSV-Incompatibility of Some Therapeutically Relevant GOIs, Limiting their Use with Conventional HAVE, and the Ability of the Hybrid HAVE Process to Circumvent this Obstacle

In simulation of the upstream-most step in the creation of rHSV vectors, V27 cells in wells of a 24-well plate were simultaneously transfected using the TransIT-X2 transfection reagent with 200 ng of genomic DNA prepared from an mCherry-encoding rHSV vector (a representative parental rHSV strain for newly constructed rHSV vectors, amongst many possible reporter-expressing and non-reporter-expressing options) and 300 ng of each of various plasmids in which a chosen GOI was placed between the CBA promoter and the rBG polyadenylation signal, and overlain the following day with medium containing 0.2% human IgG to prevent the formation of secondary plaques. Six days after transfection, plaques were stained and counted. Green fluorescent protein (GFP) is used as a control for reference, complement factor H (CFH) is representative of a transgene non-problematic with rHSV vectors, retinitis pigmentosa GTPase regulator (RPGR) and cyclic nucleotide gated channel beta 3 (CNGB3) are representative of transgenes which can be otherwise accommodated into rHSV vectors with modifications to the cassette design (e.g., use of cell-specific promoters not active during vector production), and ATP binding cassette subfamily D member 1 (ABCD1), a channel rhodopsin and GFP fusion protein (ChR-GFP), and a gap junction protein (GJP) are examples of vectors which are not able to be incorporated into rHSV vectors.

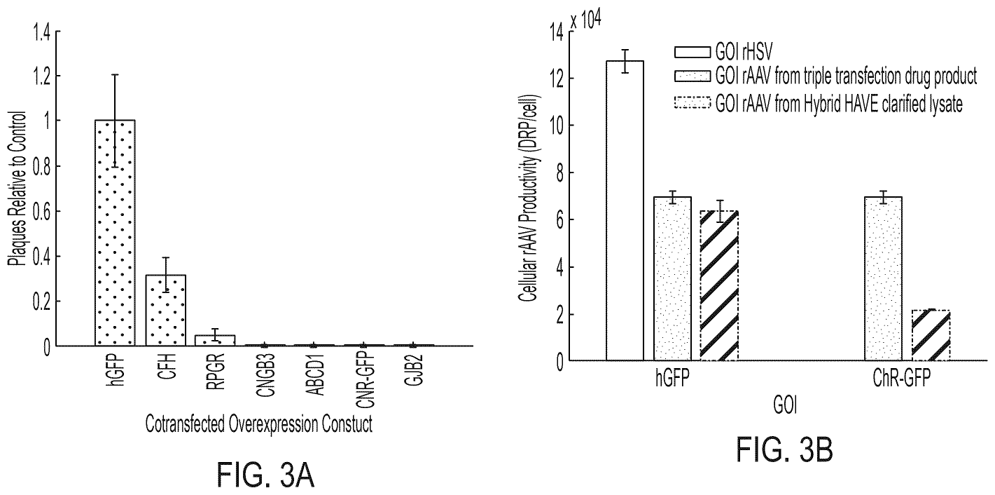

FIG. 3A shows a bar graph of plaques relative to control with respect to different cotransfected overexpression constructs, including: humanized green fluorescent protein (hGFP), complement factor H (CFH), retinitis pigmentosa GTPase regulator (RPGR), cyclic nucleotide gated channel beta 3 (CNGB3), ATP binding cassette subfamily D member 1 (ABCD1), channel rhodopsin and GFP fusion protein (ChR-GFP), and a gap junction protein (GJP). Error bar lengths represent twice the standard deviation from the mean for triplicate biological replicates.

FIG. 3B shows a bar graph of cellular rAAV productivity (DNase-resistant particles (DRP)/cell) with respect to the gene of interest (GOI), humanized green fluorescent protein (hGFP) and channel rhodopsin and GFP fusion protein (ChR-GFP). Specifically, FIG. 3B shows a demonstration of hybrid HAVE for the production of a control rAAV product (e.g., hGFP) and for the production of an otherwise problematic transmembrane GOI-encoding rAAV product at small-scale (e.g., ChR-GFP). Cultures of suspension BHK cells at a scale of 40 mL were infected with the rHSV-rep2cap2tYF vector at an MOI of 4 and the indicated GOI vector, at an MOI of 1 for the rHSV control or 100 for the rAAV. The following day, cultures were lysed via detergent and centrifuged to remove large cell debris the following day, after which vector product was quantified via a qPCR assay for DNase-resistant particles (DRP). Titered clarified lysate from an initial production using triple transfection-procured rAAV seed vector was subsequently used for a second round of expansion without significant compromise in product yield, illustrating the suitability of hybrid HAVE product for serial expansion. Error bar lengths represent twice the standard deviation from the mean for duplicate biological replicates.

Demonstration of the Robustness of the Hybrid HAVE Process at Small-Scale with Respect to Capsid Serotype Produced and Process Intermediate Used as Seed Vector

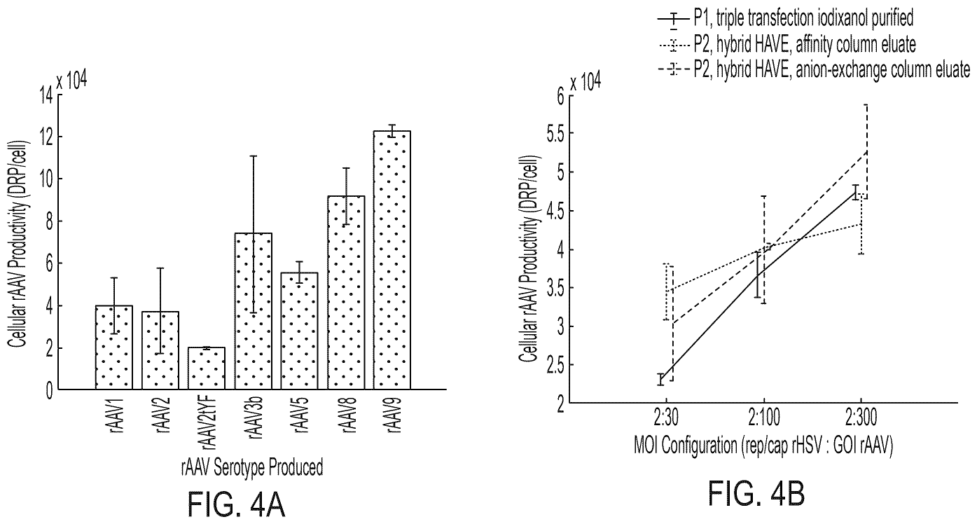

To demonstrate the ability of hybrid HAVE to produce a multitude of rAAV capsid serotypes, cultures of suspension BHK cells at a volume of 40 mL were infected with the GOI seed vector rAAV2tYF-CBA-hGFP at an MOI of 100 and each of various rep/cap rHSV stocks encoding the AAV2 rep gene and each of the indicated cap genes at an MOI of 4. The following day, cultures were lysed via detergent and centrifuged to remove large cell debris the following day, after which vector product was quantified via a qPCR assay for DNase-resistant particles (DRP). FIG. 4A shows a bar graph of cellular rAAV productivity (DNase-resistant particles (DRP)/cell) with respect to rAAV capsid serotype produced, including: rAAV1, rAAV2, rAAV2tYF, rAAV3b, rAAV5, rAAV8, and rAAV9. Error bar lengths represent twice the standard deviation from the mean for duplicate biological replicates.

To demonstrate hybrid HAVE's insensitivity to purification intermediates, rAAV production was done as for the preceding panel, but using a configuration for seed vector production entailing an (MOI of 2 for rHSV-rep2capSV6d and MOI of 100 (or a roughly half-log offset therefrom) for the GJP-encoding rAAV seed vector, comprised of P1 triple transfection-produced iodixanol gradient-purified rAAV, P2 hybrid HAVE-produced (using the aforementioned P1 rAAV) affinity column-purified rAAV (enriched for rAAV particles over other biomolecules relative to clarified lysate), or the same vector purified further through anion-exchange column (enriched for fully packaged rAAV particles over empty or capsids packaged with sub-genomic length DNA). FIG. 4B shows a graph of cellular rAAV productivity (DRP)/cell) with respect to multiplicity of infection (MOI) configuration (rep/cap rHSV: GOI rAAV). Comparable productivities across the productions with varied purification intermediates illustrate an insensitivity of the hybrid HAVE process to seed rAAV vector with diverse quality attributes and impurities (e.g., empty capsids). Error bar lengths represent twice the standard deviation from the mean for duplicate biological replicates.

Demonstration that the Hybrid HAVE Process Effectively Scales into Bioreactor-Format Manufacturing of rAAV Vector

An example of the hybrid HAVE process's capabilities for GLP-grade manufacturing of a gene therapy product in bioreactors of variable scale was captured.

FIG. 5A shows a bar graph of cellular rAAV productivity (DNase-resistant particles (DRP)/cell) with respect to bioreactor scale, e.g., at 1 L, 3 L, 10 L, and 50 L. Productions were executed in bioreactors of various formats, and cellular rAAV productivity was determined based on clarified lysate titers before processing to drug product. Error bar lengths represent twice the standard deviation from the mean for mean performances from each independent run (i.e., in bioreactor productions in which replicate bioreactors were operated concurrently, the mean productivity of said replicates was included as one replicate for the data in FIG. 5A). The impact of the rep/cap-encoding rHSV vector MOI was assessed by evaluating parallel 3 L productions with MOI configurations for rHSV and rAAV of 4 and 100 against 2 and 100, in duplicates for each. In addition to titers, profiles of rAAV packaging efficiency were compared via analytical ultracentrifugation on intermediates processed through affinity column and anion-exchange column purification steps, where proportions of empty capsids, capsids packaged with sub-vector-length DNA (“partial”), and capsids packaged with vector length DNA (full) were estimated from sedimentation analysis. Reduction of the MOI, and concomitant reduction in the depletion rate of requisite rHSV banks used for rAAV manufacture, was achieved without compromise in vector quality. FIG. 5B shows a pie chart with three components: empty capsids (“empty”), capsids packaged with sub-vector-length DNA (“partial”), and capsids packaged with vector length DNA (full), with respect to rHSV multiplicity of infection (MOI).

Head-to-Head Comparison of the Conventional HAVE, Hybrid HAVE, and Triple Transfection Processes in Bioreactor-Format rAAV Productions

To assess the performance (rAAV productivity and quality) of the hybrid HAVE process against common alternative processes, a head-to-head comparison experiment was conducted in which conventional HAVE, hybrid HAVE, and triple transfection rAAV productions were concurrently executed in 3 L bioreactors, with as many as possible common process parameters. The vector product produced in all cases was the XLRP-indicated AGTC-501 vector, rAAV2tYF-GRK1-RPGR, which expresses a transmembrane protein GOI under the control of a promoter specific to the target cell type (i.e. not expressed substantially during vector production), and is compatible with all three processes. This is achieved by: (i) for conventional HAVE, coinfecting a rep2cap2tYF-expressing rHSV vector and the GOI-encoding rHSV vector (with an MOI configuration of 2 and 1, previously optimized for this process and product); (ii) for hybrid HAVE, infecting the rep2cap2tYF-expressing rHSV vector and HAVE-manufactured AGTC-501 drug product as the GOI-encoding rAAV vector (with an MOI configuration of 2 and 100, previously optimized for production of a different product with the hybrid HAVE process); and (iii) for triple transfection, cotransfection of a plasmid expressing the same rep2cap2tYF cassette, a plasmid encoding the same GOI cassette, and a plasmid encoding requisite adenovirus helper genes (with a molar ratio of 2:2:1, previously optimized for generic triple transfection processes).

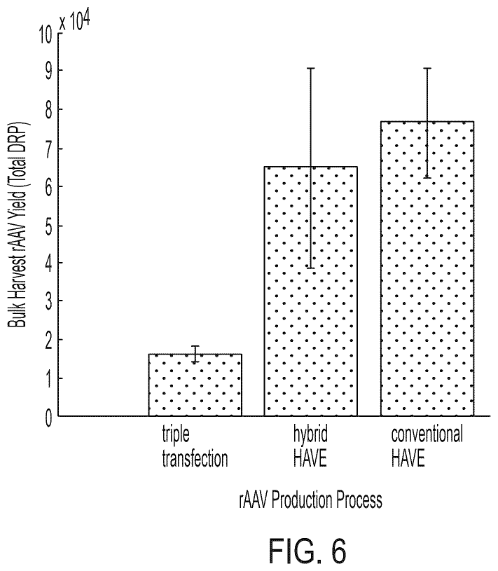

FIG. 6 shows a bar graph of the process performances with respect to total rAAV generated by the harvest time, as quantified via qPCR assay on the bulk harvest material from each process's pair of replicate reactors. Productivity trends reflect the paradigm set forth in FIG. 1D; conventional HAVE represents peak productivity, whereas hybrid HAVE compromises some productivity for versatility and ease of deployment, with both HAVE variants being superior in productivity to transfection-mediated production. Analysis of vector encapsidation efficiency was performed via AUC on affinity column eluate (i.e. starting material for anion-exchange chromatography) of bioreactor material from one replicate of each process. Relative to conventional HAVE, hybrid HAVE and triple transfection respectively showed a roughly two thirds and one tenth proportion of vector encapsidated with full length genomes.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present disclosure described herein. Such equivalents are intended to be encompassed by the following claims.

What is claimed is:

1. A method for producing recombinant adeno-associated virus (rAAV) vectors, the method comprising the steps of:
(i) providing a cell;
(ii) infecting the cell with a recombinant herpes simplex virus (rHSV) vector comprising a nucleotide sequence encoding a rep/cap gene cassette, wherein the rep/cap gene cassette comprises a adeno-associated virus serotype (AAV) rep gene and a recombinant cap gene encoding structural proteins for a predetermined capsid serotype;
(iii) infecting the cell with a recombinant adeno-associated virus (rAAV) seed vector comprising a nucleotide sequence encoding a transgene cassette, wherein the transgene cassette comprises a gene of interest (GOI);
(iv) culturing the cell under conditions that allow production of rAAV vector; and
(v) collecting the rAAV vector produced.

2. The method of claim 1, wherein:
(i) the rep/cap gene cassette comprises an AAV rep gene serotype selected from the group consisting of AAV-1, AAV-2, AAV-2tYF, AAV-3, AAV-3b, AAV-4, AAV-5, AAV-6, AAV-7, AAV-8, and AAV-9, or variants thereof;
(ii) the rep/cap gene cassette comprises a adeno-associated virus serotype 2 (AAV2) rep gene;
(iii) the predetermined capsid serotype comprises an AAV serotype selected from the group consisting of AAV-1, AAV-2, AAV-2YF, AAV-3, AAV-3b, AAV-4, AAV-5, AAV-6, AAV-7, AAV-8, and AAV-9, or variants thereof; (iv) the predetermined capsid serotype is tropic for ocular cells, retinal cells, retinal pigment epithelium, and/or photoreceptors, optionally AAV2.7m8;
(v) the predetermined capsid serotype is AAV-2 or AAV-2tYF; and/or
(vi) the predetermined capsid serotype is a non-naturally occurring, synthetic, or engineered capsid.

3. The method of any one of the preceding claims, wherein the rHSV vector is replication defective.

4. The method of any one of the preceding claims, wherein the nucleotide sequence encoding the rep/cap gene cassette is integrated into the locus of the thymidine kinase gene of the rHSV vector.

5. The method of any one of the preceding claims, wherein the rHSV vector is a HSV-1 vector.

6. The method of any one of the preceding claims, wherein the AAV rep gene,
optionally the AAV2 rep gene, is encoded by a nucleotide sequence operably linked to a nucleotide sequence encoding a promoter and/or the recombinant cap gene is encoded by a nucleotide sequence operably linked to a nucleotide sequence encoding a promoter,
optionally wherein each promoter is independently an endogenous promoter or is a heterologous promoter,
optionally wherein each promoter is independently a native AAV promoter.

7. The method of any one of the preceding claims, wherein the rAAV seed vector further comprises an additional transgene cassette, wherein the additional transgene cassette comprises a nucleotide sequence encoding a reporter molecule, optionally wherein the reporter molecule is selected from the group consisting of beta-galactosidase, neomycin phosphoro-transferase, chloramphenicol acetyl transferase, thymidine kinase, luciferase, beta-glucuronidase, xanthine-guanine phosphoribosyl transferase, and green fluorescent protein.

8. The method of any one of the preceding claims, wherein the GOI is encoded by a nucleotide sequence operably linked to a nucleotide sequence encoding a promoter, optionally wherein the promoter is an endogenous GOI promoter or is a heterologous promoter, optionally a chicken beta-actin (CBA) promoter.

9. The method of claim 7 or 8, wherein the reporter molecule is encoded by a nucleotide sequence operably linked to a nucleotide sequence encoding a promoter.

10. The method of any one of the preceding claims, wherein the transgene cassette and/or the additional transgene cassette is flanked by AAV inverted terminal repeats (ITRs), optionally AAV2 inverted terminal repeats (ITRs).

11. The method of any one of the preceding claims, wherein the transgene cassette and/or the additional transgene cassette is independently terminated by a SV40 polyadenylation signal or a bovine growth hormone polyadenylation signal.

12. The method of any one of the preceding claims, wherein the GOI encodes:
(i) a membrane protein, optionally wherein the membrane protein is selected from the group consisting of an integral membrane protein, a transmembrane protein, a peripheral membrane protein, a lipid-anchored protein, a portion thereof, and combinations thereof;
(ii) a transmembrane protein, optionally wherein the transmembrane protein comprises a light-sensing protein useful in optogenetic applications and/or optogenetic gene therapy;
(iii) a transmembrane protein, optionally wherein the transmembrane protein is selected from the group consisting of a channel protein, an ion channel protein, a transport protein, a receptor protein, a kinase protein, an adhesion protein, a structural protein, a G protein-coupled receptor, a G protein-coupled inwardly rectifying potassium channel (GIRK), a gap junction protein, a cadherin, a connexin, an opsin, a portion thereof, and combinations thereof;
(iv) an opsin, optionally wherein the opsin is selected from the group consisting of a channelrhodopsin-2 (ChR2) or an engineered variant thereof, optionally a ReaChR or ChrimsonR, a halorhodopsin (NpHR), an enhanced halorhodopsin (eNpHR), a Jaws, a rhodopsin (RHO), a short-wave cone opsin (SWC), a medium-wave cone opsin (MWC), a long-wave cone opsin (LWC), melanopsin (OPN4), an engineered opsin, optionally a Chronos (ChR90) or a multicharacteristic (polychromatic) opsin (MCO);
(v) a fusion protein; and/or
(vi) a therapeutic agent, optionally a therapeutic protein.

13. The method of any one of the preceding claims, wherein the GOI is codon-optimized for human expression.

14. The method of any one of the preceding claims, wherein the GOI is incompatible with rHSV vectors, optionally wherein:
(i) the GOI cannot be stably vectorized within rHSV; and/or
(ii) the GOI cannot be sufficiently expressed from the HSV genome.

15. The method of claim 14, wherein the GOI cannot be sufficiently expressed from the HSV genome.

16. The method of any one of the preceding claims, wherein:
(i) infection of the cell with the rHSV vector is performed before infection of the cell with the seed rAAV vector;
(ii) infection of the cell with the rHSV vector is performed after infection of the cell with the seed rAAV vector; and/or
(iii) infection of the cell with the rHSV vector is performed at about the same time as infection of the cell with the seed rAAV vector.

17. The method of any one of the preceding claims, wherein:
(i) the multiplicity of infection (MOI) of the rHSV vector is from about 1 to about 4;
(ii) the MOI of the seed rAAV vector is from about 1 to about 1,000.
(iii) the MOI configurations for the rHSV vector and the seed rAAV vector is about 4±2 and about 100±2 logs for the respective vectors; and/or
(iv) at least about 500, at least about 5000, at least about 10000, or at least about 20000, at least about 25000, at least about 30000, at least about 35000, at least about 40000, at least about 45000, at least about 50000, at least about 55000, at least about 60000, at least about 65000, at least about 70000, at least about 75000, at least about 80000, at least about 85000, at least about 90000, at least about 95000, at least about 100000 or more infectious rAAV particles are produced by the infected cell.

18. The method of any one of the preceding claims, wherein the seed rAAV vector is produced by any rAAV vector production process.

19. The method of any one of the preceding claims, wherein the seed rAAV vector is produced by a hybrid herpes-assisted vector expansion (HAVE) process and/or a transfection-based process.

20. The method of any one of the preceding claims, further comprising refeeding collected rAAV vector for recursive expansion.

21. A kit for producing rAAV comprising:
(i) a recombinant herpes simplex virus (rHSV) vector comprising a nucleotide sequence encoding a rep/cap gene cassette, wherein the rep/cap gene cassette comprises the adeno-associated virus serotype (AAV) rep gene, optionally the adeno-associated virus serotype 2 (AAV2) rep gene, and a recombinant cap gene encoding structural proteins for a predetermined capsid serotype;
(ii) a recombinant adeno-associated virus (rAAV) seed vector comprising a nucleotide sequence encoding a transgene cassette, wherein the transgene cassette comprises a gene of interest (GOI); and
(iii) instructions for use.

22. A cell, comprising:
(i) a recombinant herpes simplex virus (rHSV) vector comprising a nucleotide sequence encoding a rep/cap gene cassette, wherein the rep/cap gene cassette comprises the adeno-associated virus serotype (AAV2) rep gene, optionally adeno-associated virus serotype 2 (AAV2) rep gene, and a recombinant cap gene encoding structural proteins for a predetermined capsid serotype; and
(ii) a recombinant adeno-associated virus (rAAV) seed vector comprising a nucleotide sequence encoding a transgene cassette, wherein the transgene cassette comprises a gene of interest (GOI).

23. The cell of claim 22, which is capable of supporting infection of rHSV and delivering gene content for rAAV production.


SEQUENCE LISTING (3 sequences)

SEQ ID NO: 1 - DNA, 25 bp, synthetic construct
  source 1..25: mol_type = other DNA
agcaatagcatcacaaatttcacaa

SEQ ID NO: 2 - DNA, 29 bp, synthetic construct
  source 1..29: mol_type = other DNA
ccagacatgataagatacattgatgagtt

SEQ ID NO: 3 - DNA, 36 bp, synthetic construct
  modified_base 1: mod_base = OTHER; note = 6-carboxyfluorescein (6 FAM)
  modified_base 36: mod_base = OTHER; note = 5-carboxytetramethylrhodamine (TAMRA)
  source 1..36: mol_type = other DNA
agcatttttttcactgcattctagttgtggtttgtc